SEQUENCE LISTING (1 sequence)

SEQ ID NO: 1 - DNA, 21 bp, Artificial Sequence
  Synthetic oligonucleotide
gcaaaacaaacttattttgaa

(19) United States
(12) Patent Application Publication
(10) Pub. No.: US 2022/0185797 A1
(43) Pub. Date: Jun. 16, 2022

(54) SELECTIVE FOXO INHIBITORS FOR TREATMENT OF DIABETES AND OTHER DISORDERS RELATED TO IMPAIRED PANCREATIC FUNCTION

(71) Applicants: THE TRUSTEES OF COLUMBIA UNIVERSITYIN THE CITY OF NEW YORK, New York, NY (US); FORKHEAD BIOTHERAPEUTICS INC., Weston, MA (US)

(72) Inventors: Xiaoming XU, Fair Lawn, NJ (US); Shi-Xian DENG, White Plains, NY (US); Donald W. LANDRY, New York, NY (US); Robert J. DEVITA, Westfield, NJ (US); Hua V. LIN, Zionsville, IN (US); Yunkyoung LEE, New York, NY (US); Domenico ACCILI, New York, NY (US); Sandro BELVEDERE, Weston, MA (US)

(21) Appl. No.: 17/598,695

(22) PCT Filed: Mar. 25, 2020
(86) PCT No.: PCT/US2020/024702

§ 371 Date: Sep. 27, 2021

Related U.S. Application Data
(60) Provisional application No. 62/823,384, filed on Mar. 25, 2019.

(51) Int. Cl. C07D 403/04; A61K 31/713; A61K 31/4184; A61K 31/496; C07D 401/14; A61K 31/454; C07D 405/14; C07D 413/14; A61K 31/423; A61K 31/4709; A61K 31/4439; C07D 471/04; A61K 31/437

(52) U.S. Cl. CPC C07D 403/04; A61K 31/713; A61K 31/4184; A61K 31/496; C07D 401/14; A61K 31/454; A61K 31/437; C07D 413/14; A61K 31/423; A61K 31/4709; A61K 31/4439; C07D 471/04; C07D 405/14

(57) ABSTRACT

Various embodiments relate to a compound (represented by Formula I) or a pharmaceutically acceptable salt or tautomer thereof. The compound may selectively inhibit a Forkhead Box O1 (FOXO1) transcription factor. Various embodiments relate to methods comprising administering to a mammal having a disease or disorder associated with impaired pancreatic endocrine function, a therapeutically effective amount of the compound or a pharmaceutically acceptable salt or tautomer thereof. Various embodiments relate to methods for producing enteroendocrine cells that make and secrete insulin in a mammal, comprising administering to the mammal an effective amount of the compound or a pharmaceutically acceptable salt or tautomer thereof.

### CROSS-REFERENCE TO RELATED APPLICATION

This application claims the benefit of U.S. Provisional Patent Application No. 62/823,384, filed Mar. 25, 2019, titled SELECTIVE FOXO INHIBITORS FOR TREATMENT OF DIABETES AND OTHER DISORDERS RELATED TO IMPAIRED PANCREATIC FUNCTION, which is incorporated by reference herein in its entirety.

### FIELD OF THE INVENTION

Various embodiments of the present invention relate generally to selective FOXO inhibitors and more specifically to selective FOXO inhibitors for treatment of diabetes and other disorders related to impaired pancreatic function.

### BACKGROUND

There are more than 50 million diabetic patients worldwide that require chronic insulin treatments, including those with autoimmune type 1 diabetes and insulin-dependent type 2 diabetes. Injection of insulin is a widely used treatment with a global market size of greater than $20 billion, but it is burdensome for patients' daily lives and the health outcomes of insulin injection remain unsatisfactory. Non-invasive oral treatments for insulin dependent diabetes have the potential to improve patients' quality of life and reduce the risk for complications due to improved glycemic control. Forkhead box protein O1 also known as forkhead in rhabdomyosarcoma (FKHR) is a protein that in humans is encoded by the Forkhead Box O1 gene (FOXO1). FOXO1 is a transcription factor that plays important roles in regulation of gluconeogenesis and glycogenolysis by insulin signaling and is also central to the decision for a preadipocyte to commit to adipogenesis.

FOX (Forkhead box) proteins are a family of transcription factors. A defining feature of FOX proteins is the forkhead box, a sequence of 80 to 100 amino acids forming a motif that binds to DNA. This forkhead motif is also known as the winged helix due to the butterfly-like appearance of the loops in the protein structure of the domain. Forkhead proteins are a subgroup of the helix-turn-helix class of proteins.

Selective targeting between different Forkhead box proteins is important because the family plays important roles in regulating the expression of genes involved in cell growth, proliferation, differentiation, and longevity. It is known that selective inhibition of the transcription factor Forkhead Box O1 (FOXO1) in the gastrointestinal tract converts enteroendocrine cells into glucose-dependent insulin-producing cells. Some selective inhibitors of FOXO1 have been discovered. The FOXO1 inhibitors have the potential to be developed into a new class of drugs that reprogram gut cells into an endogenous source of insulin to replace pancreatic beta cell function and treat insulin-dependent diabetes.

A need exists, however, to discover compounds with better FOXO1 activity and selectivity and/or other useful pharmacological properties. With respect to selectivity, for example, a need exists for compounds with selective activity in favor of FOXO1 over Forkhead box protein A2 (FOXA2), which is another member of the forkhead class of DNA-binding proteins. FOXA2 serves as a transcriptional activator for liver-specific genes such as albumin and transthyretin and also plays important roles in lung and neuronal development.

### BRIEF SUMMARY

Various embodiments relate to a compound or a pharmaceutically acceptable salt or tautomer thereof. The compound may selectively inhibit a Forkhead Box O1 (FOXO1) transcription factor. Various embodiments relate to methods comprising administering to a mammal having a disease or disorder associated with impaired pancreatic endocrine function, a therapeutically effective amount of the compound or a pharmaceutically acceptable salt or tautomer thereof. Various embodiments relate to methods for producing enteroendocrine cells that make and secrete insulin in a mammal, comprising administering to the mammal an effective amount of the compound or a pharmaceutically acceptable salt or tautomer thereof.

The compound may have a structure represented by Formula I:

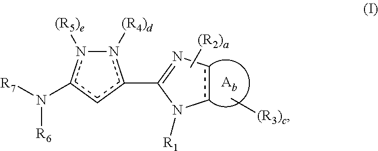

in which R₁ may be selected from the group consisting of H and C₁-C₃ alkyl; subscript “a” may be selected from the group consisting of 0, 1, and 2;
each R₂ moiety, if present, may be independently selected from the group consisting of C₁C₆ alkyl and C₃-C₁₄ aryl;
subscript “b” may be selected from the group consisting of 0 and 1;
A may be a cyclic moiety selected from the group consisting of C₃-C₁₄ aryl and C₃-C₆ heteroaryl;
subscript “c” may be selected from the group consisting of 0, 1, 2, 3, and 4;
each R₃ moiety, if present, may be independently selected from the group consisting of H, chlorine (Cl), fluorine (F), C₁-C₃ alkoxy, trifluoromethoxy (OCF₃), trifluoromethyl (CF₃), C₁-C₆ alkyl, and C₃-C₁₄ aryl;
subscript “d” may be selected from the group consisting of 0 and 1;
R₄, if present, may be selected from the group consisting of H, and C₁C₃ alkyl;
subscript “e” may be selected from the group consisting of 0 and 1;
R_{5,} if present, may be selected from the group consisting of H, and C₁-C₃ alkyl;
R₆ may be selected from the group consisting of H, and C₁C₃ alkyl;
R₇ may be selected from the group consisting of H, a moiety having a structure represented by Formula II, a moiety having a structure represented by Formula III, a moiety having a structure represented by Formula IV, a moiety having a structure represented by Formula V, a moiety having a structure represented by Formula VI, and a moiety having a structure represented by Formula VII,

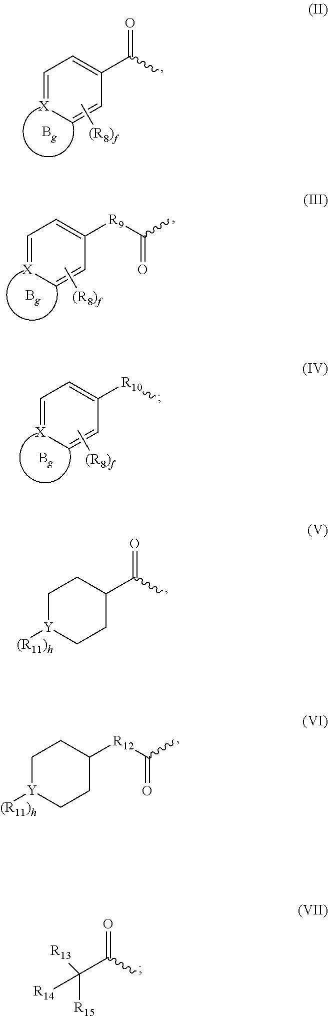

X may be selected from the group consisting of C and N;
subscript “f” may be selected from the group consisting of 3, 4, and 5;
each R₅ moiety may be independently selected from the group consisting of H, C₁-C₃ alkoxy, chlorine (Cl), fluorine (F), C₁-C₆ alkyl, trifluromethyl (CF₃), hydroxy (OH), an amine moiety, an alkyl amine moiety, an amide moiety, and a heterocyclic amine moiety;
R₉ may be a C₁-C₆ alkyl;
R₁₀ may be a C₁-C₆ alkyl;
subscript “g” may be selected from the group consisting of 0 and 1;
B may be selected from the group consisting of an aryl moiety and a heteroaryl moiety;
subscript “h” may be selected from the group consisting of 0 and 1;
R₁₁ may be selected from the group consisting of H, C₁-C₆ alkyl, and C₁-C₃ alkoxy;
R₁₂ may be a C₁-C₆ alkyl;
Y may be selected from the group consisting of C, N, and O;
R₁₃ may be a C₁-C₆ alkyl;
R₁₄ may be a C₁-C₆ alkyl; and
R₁₅ may be a C₁-C₆ alkyl.
Various embodiments may exclude Compound 1 and Compound 2:

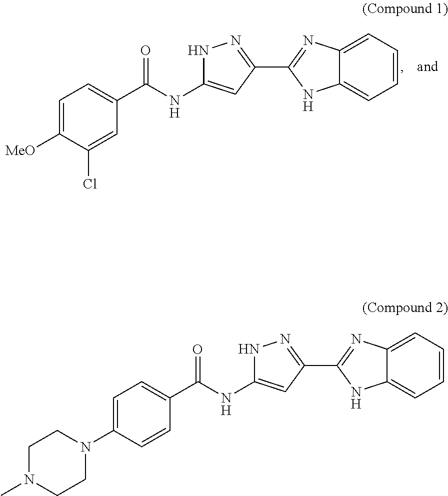 [SMILES: COc1ccc(C(=O)Nc2cc(-c3nc4ccccc4[nH]3)n[nH]2)cc1Cl ; CN1CCN(c2ccc(C(=O)Nc3cc(-c4nc5ccccc5[nH]4)n[nH]3)cc2)CC1]

### DETAILED DESCRIPTION

Introduction and Definitions

Various embodiments may be understood more readily by reference to the following detailed description. Unless defined otherwise, all technical and scientific terms used herein have the same meaning as commonly understood by one of ordinary skill in the art to which this disclosure belongs.

As used herein, the term “standard temperature and pressure” generally refers to 20° C. and 1 atmosphere. Standard temperature and pressure may also be referred to as “ambient conditions.” Unless indicated otherwise, parts are by weight, temperature is in ° C., and pressure is at or near atmospheric. The terms “elevated temperatures” or “high-temperatures” generally refer to temperatures of at least 100° C.

The term “mol percent” or “mole percent” generally refers to the percentage that the moles of a particular component are of the total moles that are in a mixture. The sum of the mole fractions for each component in a solution is equal to 1.

“An active agent” means a small molecule compound described herein that causes any Ins− cell, enteroendocrine cell such as serotonin, Tph1 or somatostatin-expressing cells, or Neurogenin3 progenitor in the gut to differentiate into an Ins+cell. Certain active agents are those that reduce the expression, biosynthesis, signaling or biological activity of FOXO1. Active agents include prodrug versions of the small molecule compound embodiments.

“Conjunctive agent” as used herein refers to an agent other than an active agent that has therapeutic activity related to a target disease or disorder. A conjunctive agent may inhibit Foxo (e.g. FOXO1) or is an agent known to treat or prevent a pathology associated with impaired pancreatic function. Examples of conjunctive agents include but are not limited to inhibitory oligonucleotides that reduce expression of a Foxo gene or Foxo protein (See: U.S. Pat. Nos. 9,457,079 and 8,580,948), antibodies targeting a Foxo gene or Foxo protein (e.g. Foxo1); or drugs known to treat pathology associated with pancreatic function such as metformin, sulfonylureas, meglitinides, thiazolidenediones, DDP-4 inhibitors, GLP-1 receptor agonists, SGLT2 inhibitors and insulin.

“Preventing a disease” includes, but is not limited to, preventing the disease from occurring in a subject that may be predisposed to the disease (or disorder), but has not yet been diagnosed as having the disease; inhibiting the disease, for example, arresting the development of the disease; relieving the disease, for example by causing its regression; relieving the condition caused by the disease, for example by reducing its symptoms, and/or delaying disease onset. An example is reducing blood glucose levels in a hyperglycemic subject, and/or maintaining acceptable control of blood glucose levels in the subject. Such treatment, prevention, symptoms and/or conditions can be determined by one skilled in the art and are described in standard textbooks.

“Treating” a disease, disorder or condition in a patient refers to taking steps to obtain beneficial or desired results, including clinical results. For purposes of this invention, beneficial or desired clinical results include, but are not limited to alleviation or amelioration of one or more symptoms of the disease; diminishing the extent of disease; delaying or slowing disease progression; amelioration and palliation or stabilization of the disease state.

Where the disease is diabetes type 1, symptoms include frequent urination, excessive thirst, extreme hunger, unusual weight loss, increased fatigue, irritability, blurry vision, genital itching, odd aches and pains, dry mouth, dry or itchy skin, impotence, vaginal yeast infections, poor healing of cuts and scrapes, excessive or unusual infections. These symptoms are associated with characteristic clinical laboratory findings that include hyperglycemia (excessively elevated sugar concentrations in the blood, i.e. >125 mg/dl), loss of glycemic control (i.e., frequent and excessive swings of blood sugar levels above and below the physiological range, generally maintained between 60-125 mg/dl), fluctuations in postprandial blood glucose, fluctuations in blood glucagon, fluctuations in blood triglycerides and include reduction in rate of or diminution of or improved outcomes of conditions that are accelerated by and/or occur because of or more frequently with diabetes including microvascular and microvascular disease inclusive but not limited to cerebrovascular impairment with or without, stroke, angina, coronary heart disease, myocardial infarction, peripheral vascular disease, nephropathy, kidney impairment, increased proteinuria, retinopathy, neovascularization of vessels in the retina, neuropathy including central, autonomic and peripheral neuropathy that may lead to loss of sensation of extremities and amputation and/or from neuropathy or diminished vascular flow, skin conditions including but not limited to diabetic dermopathy, necrobiosis lipoidica diabeticorum, bullosis diabeticorum, scleroderma diabeticorum, granuloma annulare, bacterial skin infections (including but limited to Staphylococcus, which can result in deeper infections), periodontal disease, and gastroparesis (abnormal emptying of the stomach). Type 1 diabetes may be diagnosed by methods well known to one of ordinary skill in the art. For example, commonly, diabetics have a plasma fasting blood glucose result of greater than 126 mg/dL of glucose. Prediabetes is commonly diagnosed in patients with a blood glucose level between 100 and 125 mg/dL of glucose. Other symptoms may also be used to diagnose diabetes, related diseases and conditions, and diseases and conditions affected by diminished pancreatic function.

“Reduction” of a symptom(s) means a decreasing of the severity or frequency of the symptom(s), or elimination of the symptom(s).

“Pathology associated with impaired pancreatic function” or pancreatic malfunction is one in which the pathology is associated with a diminished capacity in a subject for the pancreas to produce and/or secrete one or more pancreatic hormones including insulin and/or pancreatic peptides such as glucagon, pancreatic polypeptide, or somatostatin. Pathologies that are associated with impaired pancreatic function include type 1 diabetes, and type 2 diabetes. Other pathologies include those sometimes referred to as latent autoimmune diabetes of adulthood, pre-diabetes, impaired fasting glucose, impaired glucose tolerance, fasting hyperglycemia, insulin resistant syndrome, insulin secretion deficiency secondary to a partial or total pancreatectomy, and hyperglycemic conditions.

“Administering” or “administration of” a drug or therapeutic pharmaceutical composition to a subject by any method known in the art includes both direct administration, including self-administration (including oral administration or intravenous, subcutaneous, intramuscular or intraperitoneal injections, rectal administration by way of suppositories), local administration directly into or onto a target tissue (such as a region of the gut that has gut ins- cells), or administration by any route or method that delivers a therapeutically effective amount of the drug or composition to the cells or tissue to which it is targeted.

As used herein, the terms “co-administered, “co-administering,” or “concurrent administration”, when used, for example with respect to administration of an active agent with another active agent, or a conjunctive agent along with administration of an active agent refers to administration of the active agent and the other active agent and/or conjunctive agent such that both can simultaneously achieve a physiological effect. The two agents, however, need not be administered together. In certain embodiments, administration of one agent can precede administration of the other, however, such co-administering typically results in both agents being simultaneously present in the body (e.g. in the plasma) of the subject. Co-administering includes providing a co-formulation (in which the agents are combined or compounded into a single dosage form suitable for oral, subcutaneous or parenteral administration).

A “subject” or “patient” is a mammal, typically a human, but optionally a mammalian animal of veterinary importance, including but not limited to horses, cattle, sheep, dogs, and cats.

A “therapeutically effective amount” of an active agent or pharmaceutical composition is an amount that achieves the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

A “prophylactically effective amount” of a drug is an amount of a drug that, when administered to a subject, will have the intended prophylactic effect, e.g., preventing or delaying the onset (or reoccurrence) of the disease or symptoms, or reducing the likelihood of the onset (or reoccurrence) of the disease or symptoms. The full prophylactic effect does not necessarily occur by administration of one dose and may occur only after administration of a series of doses. Thus, a prophylactically effective amount may be administered in one or more administrations. For diabetes, a therapeutically effective amount can also be an amount that increases insulin secretion, increases insulin sensitivity, increases glucose tolerance, or decreases weight gain, weight loss, or fat mass.

An “effective amount” of an agent is an amount that produces the desired effect.

By “pharmaceutically acceptable” is meant that the carrier, diluent or excipient must be compatible with the other ingredients of the formulation and not deleterious to the recipient thereof.

“Foxo Protein” includes FOXO1, FOXO3, FOXO4 and FOXO6 from human, and Foxo1, 3, 4, and 6 proteins from other mammals, including variants, orthologs, and biologically active fragments thereof.

“Foxo gene” means any gene encoding a Foxo protein, including orthologs, and biologically active fragments thereof.

“Foxo mRNA” means any mRNA encoding a Foxo protein, including orthologs, and biologically active fragments thereof.

“Gut Ins⁻ cell(s)” and “gut ins-negative cell(s)” are used interchangeably herein and broadly refers to any non-insulin producing cell in the gut. Enteroendocrine cells that do not express insulin are a subset of gut Ins⁻ cells. Terminally differentiated cells in the gut that do not produce insulin are also gut Ins⁻ cells.

“Gut Ins⁺ cell(s)” broadly means any cell in the gut that has differentiated into an Insulin⁺ cell in response to contact with an active agent as described herein. Ins⁺ enteroendocrine cells are a subset of gut Ins⁺ cells as are any Ins⁺ cell in the gut that have differentiated in response to contact with an active agent as described herein.

“Enteroendocrine cells” means specialized Insulin-negative endocrine cells in the gastrointestinal tract. Enteroendocrine cells (a subset of Gut Ins⁻ cells) produce various one or more other hormones such as gastrin, ghrelin, neuropeptide Y, peptide YY₃₋₃₆ (PYY₃₋₃₆), serotonin, secretin, somatostatin, motilin, cholecystokinin, gastric inhibitory peptide, neurotensin, vasoactive intestinal peptide, glucose-dependent insulinotropic polypeptide (GIP) or glucagon-like peptide-1. Enteroendocrine cells and any other gut insulin-negative cell capable of differentiating into an insulin-positive cell are the targets of the active agents of the invention.

“Insulin-producing enteroendocrine cells” mean any enteroendocrine cells that make and secrete insulin; they are a subset of Gut Ins⁺ cells. Insulin-producing enteroendocrine cells have the insulin positive phenotype (Ins⁺) so that they express markers of mature beta-cells, and secrete insulin and C-peptide in response to glucose and sulfonylureas. Insulin-producing enteroendocrine cells arise primarily from neurogenin-3 (N3)Prog and also from gut stem cells.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

Unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

The examples and embodiments described herein are for illustrative purposes only and that various modifications or changes in light thereof will be suggested to persons skilled in the art and are to be included within the spirit and purview of this application. Many variations and modifications may be made to the above-described embodiment(s) of the disclosure without departing substantially from the spirit and principles of the disclosure. All such modifications and variations are intended to be included herein within the scope of this disclosure.

Various embodiments are described by reference to chemical structures. In the chemical structures, various chemical moieties are represented by R-groups. Some R-groups are described by reference to another chemical structure. A wavy bond line in a structure representing an R-group indicates the point at which the R-group is attached to or bonded to the main structure. In some chemical structures various cyclic moieties are represented by lettered rings. The lettered ring may represent a variety of cyclic structures. Some cyclic structures are described by reference to another chemical structure. A wavy bond line in a structure representing a cyclic structure indicates a bond that is shared with the main structure, or the point at which the cyclic structure is fused, attached, joined, or bonded to the main structure to form a polycyclic structure. Various subscripts are also used. Each R-group has a numeric subscript which distinguishes it from other R-groups. R-groups and lettered rings may also include a lowercase alphabetical subscript, indicating that different embodiments, may have differing numbers of that moiety. If a lowercase alphabetical subscript may be 0, it means that, in some embodiments, the moiety may not be present. A dashed line in a cyclic structure indicates that in various embodiments one or more double-bounds may be present. When a compound may include more than one instance of a moiety, for example a moiety represented by an R-group, and that moiety is described as being “independently selected” from a list of options, each instance may be selected from the complete list without respect to any prior selections from the list; in other words, the instances may be the same or different and the same list item may be selected for multiple instances. Some R-group substitutions indicate a range, such as C₁-C₆ alkyl. Such a range indicates that the R-group may be a C₁ alkyl, a C₂ alkyl, a C₃ alkyl, a C₄ alkyl, a C₅ alkyl, or a C₆ alkyl. In other words, all such ranges are intended to include an explicit reference to each member within the range.

### Chemical Definitions:

The term “alkyl” as used herein alone or as part of another group refers to any saturated aliphatic hydrocarbon, including straight-chain and branched-chain alkyl groups. In one embodiment the alkyl group has 1-6 carbons designated here as C₁-C₆-alkyl. In another embodiment, the alkyl group has 1-3 carbons designated here as C₁-C₃-alkyl.

The term “aryl” used herein alone or as part of another group refers to an aromatic ring system containing from 3 to 14 ring carbon atoms. In some embodiments, the term aryl refers to an aromatic ring system containing from 3-6 ring carbon atoms. In other embodiments, the term aryl refers to an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. A currently preferred aryl group is phenyl. Non-limiting examples of aryl include phenyl (a C₆ aryl).

The term “heteroaryl” as used herein alone or as part of another group refers to a heteroaromatic system containing at least one heteroatom ring wherein the atom is selected from nitrogen, sulfur and oxygen. In some embodiments, the heteroaryl contains 3 or more ring atoms. In some embodiments, the heteroaryl contains 3-6 ring carbon atoms (C₃-C₆ heteroaryl). In some embodiments, the heteroaryl contains 5 or more ring atoms. The heteroaryl group can be monocyclic, bicyclic, tricyclic and the like. Also included in this definition are the benzoheterocyclic rings. If nitrogen is a ring atom, the present invention also contemplates the N-oxides of the nitrogen containing heteroaryls. Non-limiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like.

Non-limiting examples of C₃-C₆ heteroaryl include pyrrolyl, pyridinyl, imidazolyl, pyrazolyl, oxazolyl, isoxazolyl, thiophenyl, furyl, thiazolyl, and isothiazolyl.

The term “heterocyclic ring” or “heterocyclyl” as used herein alone or as part of another group refers to a five-membered to eight-membered rings that have 1 to 4 heteroatoms, such as oxygen, sulfur and/or nitrogen, in particular nitrogen, either alone or in conjunction with sulfur or oxygen ring atoms. These five-membered to eight-membered rings can be saturated, fully unsaturated or partially unsaturated, with fully saturated rings being preferred. Preferred heterocyclic rings include piperidinyl, pyrrolidinyl pyrrolinyl, pyrazolinyl, pyrazolidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, dihydrothiazolyl, succinimidnyl, maledimidyl, and the like. Non-limiting examples of currently preferred heterocyclic groups include pyrrole, pyrrolidine, piperidine, succinimide, maleimide, morpholine, tetrahydrofuran, pyran, and tetrahydropyran.

The term “hydroxy” as used herein alone or as part of another group refers to an OH group.

The term “alkoxy” as used herein alone or as part of another group refers to an —O-alkyl group wherein alkyl is as defined above. As used herein C₁-C₃ alkoxy may refer to methoxy, ethoxy, propoxy, or isopropoxy.

The term “amine” as used herein alone or as part of another group refers to an NRR′ group wherein each of R and R′ independently is H or alkyl as defined above.

The term “amide” as used herein alone or as part of another group refers to a —C(O)NRR′ group wherein each of R and R′ independently is H or alkyl as defined above.

The term “halogen” or “halo” as used herein alone or as part of another group refers to chlorine, bromine, fluorine, and iodine. The term “haloalkyl” refers to an alkyl group having some or all of the hydrogens independently replaced by a halogen group including, but not limited to, trichloromethyl, tribromomethyl, trifluoromethyl, triiodomethyl, difluoromethyl, chlorodifluoromethyl, pentafluoroethyl, 1,1-difluoroethyl bromomethyl, chloromethyl, fluoromethyl, iodomethyl, and the like. A currently preferred haloalkyl group is triluoromethyl (CF₃).

As used herein, the symbol “ [symbol] ” designates a point of attachment of a particular substituents to the rest of the molecule.

General Discussion of Active Agents

Various embodiments of active agents relate to compounds, which may selectively inhibit a Forkhead Box O1 (FOXO1) transcription factor (human or other non-human mammals), and which may have a structure represented by Formula I:

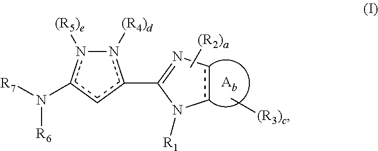

in which R₁ may be selected from the group consisting of H and C₁-C₃ alkyl;
the subscript “a” may be selected from the group consisting of 0, 1, and 2;
each R₂ moiety, if present, may be independently selected from the group consisting of C₁-C₆ alkyl and C₃-C₁₄ aryl;
the subscript “b” may be selected from the group consisting of 0 and 1;
A may be a cyclic moiety selected from the group consisting of C₃-C14 aryl and C₃-C₆ heteroaryl;
the subscript “c” may be selected from the group consisting of 0, 1, 2, 3, and 4;
each R₃ moiety, if present, may be independently selected from the group consisting of H, chlorine (Cl), fluorine (F), C₁-C₃ alkoxy, trifluoromethoxy (OCF₃), trifluoromethyl (CF₃), C₁-C₆ alkyl, and C₃-C₁₄ aryl;
the subscript “d” may be selected from the group consisting of 0 and 1;
if present, R₄ may be selected from the group consisting of H, and C₁-C₃ alkyl;
the subscript “e” may be selected from the group consisting of 0 and 1;
if present, R₅ may be selected from the group consisting of H, and C₁-C₃ alkyl;
R₆ may be selected from the group consisting of H, and C₁-C₃ alkyl;
R₇ may be selected from the group consisting of H, a moiety having a structure represented by Formula II, a moiety having a structure represented by Formula III, a moiety having a structure represented by Formula IV, a moiety having a structure represented by Formula V, a moiety having a structure represented by Formula VI, and a moiety having a structure represented by Formula VII,

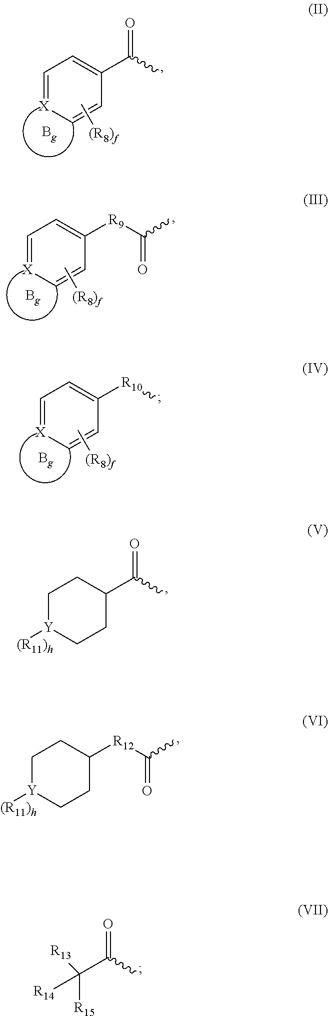

in which X may be selected from the group consisting of carbon (C) and nitrogen (N);
the subscript “f” may be selected from the group consisting of 3, 4, and 5;
each R₈ moiety may be independently selected from the group consisting of H, C₁-C₃ alkoxy, chlorine (Cl), fluorine (F), C₁-C₆ alkyl, trifluoromethyl (CF₃), hydroxy (OH), an amine moiety, an alkyl amine moiety, an amide moiety, and a heterocyclic amine moiety;
R₉ may be a C₁-C₆ alkyl;
R₁₀ may be a C₁-C₆ alkyl;
the subscript “g” may be selected from the group consisting of 0 and 1;
B may be selected from the group consisting of an aryl moiety and a heteroaryl moiety;
the subscript “h” may be selected from the group consisting of 0 and 1;
R₁₁ may be selected from the group consisting of H, C₁-C₆ alkyl, and C₁-C₃ alkoxy;
R₁₂ may be a C₁-C₆ alkyl;
Y may be selected from the group consisting of carbon (C), nitrogen (N), and oxygen (O);
R₁₃ may be a C₁-C₆ alkyl;
R₁₄ may be a C₁-C₆ alkyl; and
R₁₅ may be a C₁-C₆ alkyl
or a pharmaceutically acceptable salt or tautomer thereof.

According to various embodiments A may be a pyridine moiety. For example, according to some embodiments, the pyridine moiety may be selected from the group consisting of a moiety having a structure represented by Formula VIII and a moiety having a structure represented by Formula IX,

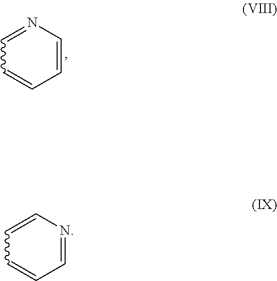 [SMILES: c1ccncc1 ; c1ccncc1]

According to various embodiments, R₈ may be an amine moiety, as indicated above, and the amine moiety may have a structure represented by Formula X

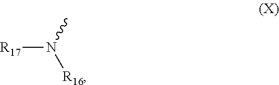

in which R₁₆ may be selected from the group consisting of H and C₁-C₃ alkyl; and
R₁₇ may be selected from the group consisting of H and C₁-C₃ alkyl.

According to various embodiments, R₈ may be an alkyl amine moiety, as indicated above, and the alkyl amine moiety may have a structure represented by Formula XI

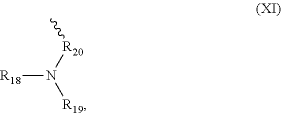

in which R₁₈ may be selected from the group consisting of H and C₁-C₃ alkyl; R₁₉ may be selected from the group consisting of H and C₁-C₃ alkyl; and R₂₀ may be a C₁-C₆ alkyl.

According to various embodiments, R₈ may be an amide moiety, as indicated above, and the amide moiety may have a structure represented by Formula XII

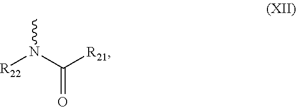

in which R₂₁ may be selected from the group consisting of H and C₁-C₃ alkyl; and
R₂₂ may be selected from the group consisting of H and C₁-C₃ alkyl.

According to various embodiments, R₈ may be a heterocyclic amine moiety, as indicated above, and the heterocyclic amine moiety may have a structure represented by Formula XIII

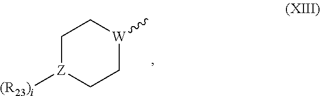

in which, the subscript “i” may be selected from the group consisting of 0 and 1; if present, R₂₃ may be selected from the group consisting of H, C₁-C₆ alkyl, and a ketone moiety; Z may be selected from the group consisting of carbon (C), nitrogen (N), and oxygen (O); W may be selected from the group consisting of carbon (C) and nitrogen (N).

According to various embodiments, R₈ may be a heterocyclic amine moiety, as indicated above, and the heterocyclic amine moiety may have a structure represented by Formula XIV

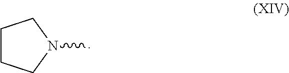

With reference to Formula II, Formula III, and Formula IV, the subscript “g” may be 1, indicating that a cyclic moiety B is present. The cyclic moiety B may be the heteroaryl moiety, and the heteroaryl moiety may be selected from the group consisting of a moiety having a structure represented by Formula XV,

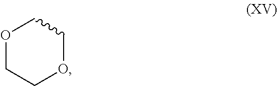

a moiety having a structure represented by Formula XVI,

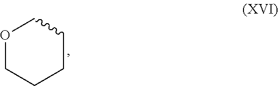

a moiety having a structure represented by Formula XVII,

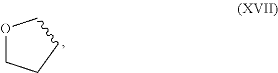

a moiety having a structure represented by Formula XVIII,

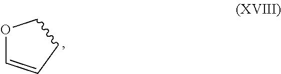

a moiety having a structure represented by Formula XIX,

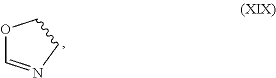

a moiety having a structure represented by Formula XX,

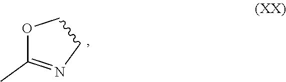

a moiety having a structure represented by Formula XXI,

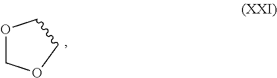

a moiety having a structure represented by Formula XXII,

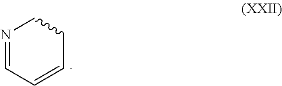

According to certain embodiments, the compound may have an IC₅₀ less than or equal to 50 nM and a maximal inhibition of FOXO1 of greater than 40%. For example, in certain embodiments, R₁ is H; a is 0; b is 1; A is a C₆ aryl (e.g., phenyl); c is 4; each R₃ moiety is independently selected from the group consisting of H, chlorine, and methoxy; d is selected from the group consisting of 0 and 1; if present, R₄ is selected from the group consisting of H, and C₁-C₃ alkyl; e is selected from the group consisting of 0 and 1; if present, R₅ is H; R₆ is H; R₇ is a moiety having a structure represented by Formula II;

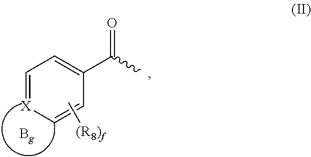

in which, g is 0; f is 5; and each R₈ moiety is independently selected from the group consisting of H, C₁-C₃ alkoxy, chlorine (Cl), the amine moiety, and the heterocyclic amine moiety. In certain embodiments, the amine moiety may have a structure represented by Formula X

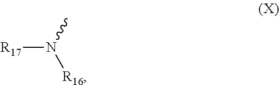

in which R₁₆ is a C₁-C₂ alkyl; and R₁₇ is a C₁-C₂ alkyl. In certain embodiments, the heterocyclic amine moiety may have a structure represented by Formula XIII

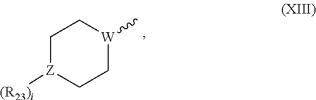

in which i is selected from the group consisting of 0 and 1; if present, R₂₃ is selected from the group consisting of H, and C₁ alkyl; Z is selected from the group consisting of carbon (C), nitrogen (N), and oxygen (O); and W is nitrogen (N). In certain embodiments, the heterocyclic amine moiety may have a structure represented by Formula XIV

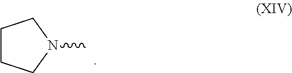

According to various embodiments, R₇ may be a moiety represented by Formula II, wherein X is C, g is 0 and f is 5. Each Rs moiety may be independently selected from the group consisting of H, C₁-C₃ alkoxy, fluorine (F), C₁-C₆ alkyl, trifluromethyl (CF₃), hydroxy (OH), an amine moiety, an alkyl amine moiety, an amide moiety, or a heterocyclic amine moiety. A may be phenyl and b may be 1. c may be selected from the group consisting of 1, 2, 3, or 4. a maybe 1 or 2.

According to various embodiments, R₇ may be a moiety represented by Formula II, wherein X is C, g is 0 and f is 5. Each R₈ moiety may be independently selected from the group consisting of H, C₂-C₃ alkoxy, chlorine (Cl), fluorine (F), C₁-C₆ alkyl, trifluromethyl (CF₃), hydroxy (OH), an amine moiety, an alkyl amine moiety, an amide moiety, or a heterocyclic amine moiety.

According to various embodiments, R₇ may be a moiety represented by Formula II, wherein X is C, g is 0 and f is 5. Rs moiety may be independently selected from the group consisting of H, chlorine (Cl), fluorine (F), C₁-C₆ alkyl, trifluromethyl (CF₃), hydroxy (OH), an amine moiety, an alkyl amine moiety, an amide moiety, and a heterocyclic amine moiety.

According to various embodiments, R₇ may be a moiety represented by Formula II, wherein X is C, g is 0 and f is 5. Rs moiety may be independently selected from the group consisting of H, C₁-C₃ alkoxy, chlorine (Cl), fluorine (F), C₁-C₆ alkyl, trifluromethyl (CF₃), hydroxy (OH), an amine moiety, and an alkyl amine moiety.

According to various embodiments, one of R₄ and R₅ may be methyl.

Various embodiments relate to pharmaceutical compositions comprising a compound according to any of the embodiments described herein or a pharmaceutically acceptable salt or tautomer thereof. For example, various embodiments may relate to a pharmaceutical composition comprising a compound having a structure represented by formula I:

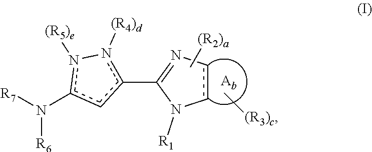

and a pharmaceutically-acceptable excipient in a unit dosage form, wherein R₁ is selected from the group consisting of H and C₁-C₃ alkyl;
wherein a is selected from the group consisting of 0, 1, and 2;
wherein each R₂ moiety, if present, is independently selected from the group consisting of C₁-C₆ alkyl
and C₃-C₁₄ aryl;
wherein b is selected from the group consisting of 0 and 1;
wherein A is a cyclic moiety selected from the group consisting of C₃-C₁₄ aryl and C₃-C₆ heteroaryl;
wherein c is selected from the group consisting of 0, 1, 2, 3, and 4;
wherein each R₃ moiety, if present, is independently selected from the group consisting of H, chlorine (Cl), fluorine (F), C₁-C₃ alkoxy, trifluoromethoxy (OCF₃), trifluoromethyl (CF₃),
C₁-C₆ alkyl, and C₃-C₁₄ aryl;
wherein d is selected from the group consisting of 0 and 1;
wherein, if present, R₄ is selected from the group consisting of H, and C₁-C₃ alkyl;
wherein e is selected from the group consisting of 0 and 1;
wherein, if present, R₅ is selected from the group consisting of H, and C₁-C₃ alkyl;
wherein R₆ is selected from the group consisting of H, and C₁-C₃ alkyl;
wherein R₇ is selected from the group consisting of H, a moiety having a structure represented by Formula II, a moiety having a structure represented by Formula III, a moiety having a structure represented by Formula IV, a moiety having a structure represented by Formula V, a moiety having a structure represented by Formula VI, and a moiety having a structure represented by Formula VII,

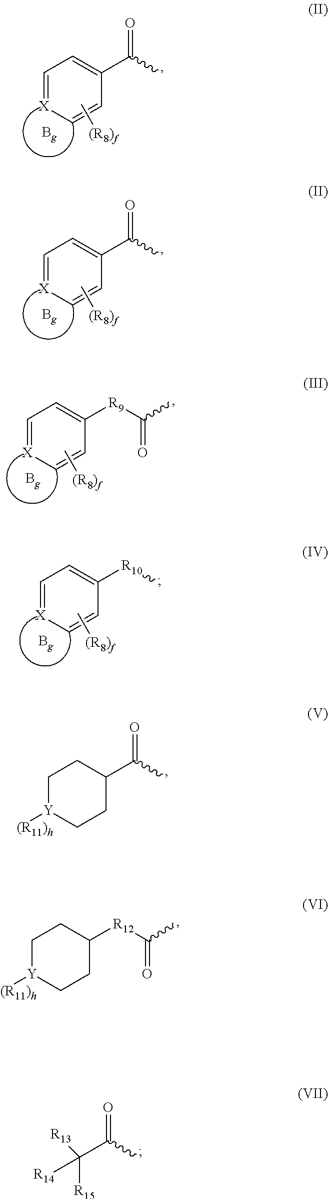

wherein X is selected from the group consisting of C and N;
wherein f is selected from the group consisting of 3, 4, and 5;
wherein each R₈ moiety is independently selected from the group consisting of H, C₁-C₃ alkoxy, chlorine (Cl), fluorine (F), C₁-C₆ alkyl, trifluromethyl (CF₃), hydroxy (OH), an amine moiety, an alkyl amine moiety, an amide moiety, and a heterocyclic amine moiety;
wherein R₉ is a C₁-C₆ alkyl;
wherein R₁₀ is a C₁-C₆ alkyl;
wherein g is selected from the group consisting of 0 and 1;
wherein B is selected from the group consisting of an aryl moiety and a heteroaryl moiety;
wherein h is selected from the group consisting of 0 and 1;
wherein R₁₁ is selected from the group consisting of H, C₁-C₆ alkyl, and C₁-C₃ alkoxy;
wherein R₁₂ is a C₁-C₆ alkyl;
wherein Y is selected from the group consisting of C, N, and O;
wherein R₁₃ is a C₁-C₆ alkyl;
wherein R₁₄ is a C₁-C₆ alkyl; and
wherein R₁₅ is a C₁-C₆ alky,
   or a pharmaceutically acceptable salt or tautomer thereof.

In some embodiments, the pharmaceutical compositions further comprises at least one pharmaceutically acceptable carrier or excipient.

Various embodiments relate to an orally-administered medicament for the treatment of diabetes, the medicament comprising one or more compounds as described above.

Various embodiments relate to a method of treating insulin-dependent type 2 diabetes, the method may include administering an effective dosage of one or more of the compounds, as described above, to a patient. The one or more compounds may selectively inhibit a Forkhead Box O1 (FOXO1) transcription factor.

According to various embodiments, certain compounds may be excluded. For example, according to some embodiments,

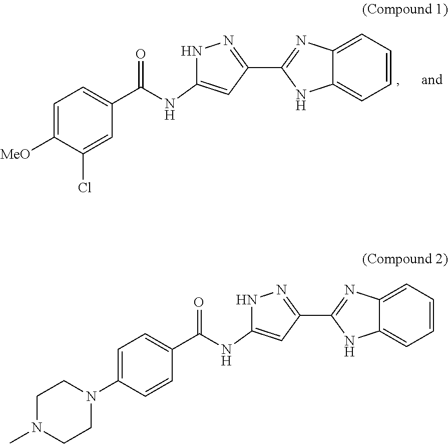 [SMILES: COc1ccc(C(=O)Nc2cc(-c3nc4ccccc4[nH]3)n[nH]2)cc1Cl ; CN1CCN(c2ccc(C(=O)Nc3cc(-c4nc5ccccc5[nH]4)n[nH]3)cc2)CC1]

and tautomers thereof, may be excluded. Similarly, according to various embodiments compounds resulting from certain combinations of substituents may be excluded. For example, compounds of formula (I) when

R₁ is H;

a is 0;

A is phenyl;

b is 1;

c is 0, i.e., the phenyl ring is unsubstituted (or alternatively, c is 4 and R₃ is H at each occurrence);

d is 0, e is 1 and R₅ is H, or d is 1, R₄ is H and e is 0;

R₆ is H;

R₇ is a moiety represented by Formula II

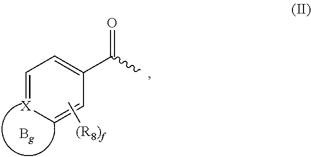

X is C, g is 0 and f is 5,

compounds in which R₈ is a methoxy at position 4 of the phenyl ring, a chloro position at position 3 of the phenyl ring, and H at all other positions; or R₈ is an N-methylpiperazinyl at position 4 of the phenyl ring, and H at all other positions, may be excluded.

### Therapeutic Methods

U.S Pat. No. 9,457,079 ('079 Patent), and US Pat. Pub 2017/0204375 ('375 pub) are incorporated herein in their entirety and describe a number of therapeutic uses for implementing Foxo inhibitors to treat impaired pancreatic function disorders. Namely, the patent discusses the use of Foxo1 inhibitors for the purpose of generating insulin-positive enteroendocrine cells in the gut. Accordingly, certain embodiments of the invention are directed to methods for producing mammalian Gut Ins⁺ cells by contacting gut cells with an Foxo inhibitor embodiment described herein that causes the cells to become Gut Ins⁺ cells. Preferred agents include those that reduce expression of one or more Foxo genes or mRNA encoding one or more Foxo proteins, or reduce the biological activity of one or more Foxo proteins to a level that permits the gut ins⁻ cell to convert into cells having the Gut Ins⁺ cells phenotype. The Gut Ins⁻ cells can be contacted with the agent in situ in the animal, or enriched populations of Gut Ins⁻ can be isolated from the gut, or intestinal explants in culture can be used. Some of these methods are described in Example 10 of '079 Patent. Certain other embodiments are directed to the isolated Gut Ins⁺ cells themselves, and to tissue explants that include Gut Ins⁺ cells, preferably intestinal tissue but artificial tissues are also included. Additional methods include the generation of Ins⁺ cells from cells that have been reprogrammed in vitro to become gut N3 prog or other gut ins- cells; in other words, gut N3 cells that have been obtained indirectly through manipulation of other cell types. For example, others made insulin-producing cells from skin biopsies by “reprogramming” cells.

These methods and others known in the art can be used in the embodiments of the invention. Maehr R, et al., 2009 Proc Natl Sci Acad USA 106(37):15768-73; Epub 2009 Aug. 31, Generation of pluripotent stem cells from patients with type 1 diabetes.

Efficacy of the methods of treatment described herein can be monitored by determining whether the methods ameliorate any of the symptoms of the disease being treated. Alternatively, one can monitor the level of serum insulin or C-peptide (a byproduct of insulin secretion and an index of functional Ins+ cells), which levels should increase in response to therapy. Alternatively, efficacy can be measured by monitoring glycemia, glucose tolerance, fat mass, weight gain, ketone bodies or other indicia of the enumerated disease or disorder in the subject being treated.

In addition to reduced insulin secretion, impaired pancreatic function includes an altered capacity to produce and/or secrete one or more pancreatic hormones including one or more pancreatic peptides such as glucagon, islet amyloid polypeptide (IAPP), pancreatic polypeptide, somatostatin, or ghrelin. Well known pathologies that are associated with impaired pancreatic function include type 1 diabetes and type 2 diabetes. Other pathologies include those sometimes referred to as latent autoimmune diabetes of adulthood, pre-diabetes, impaired fasting glucose, impaired glucose tolerance, fasting hyperglycemia, insulin resistant syndrome, and hyperglycemic conditions. All of these come within the meaning of treating and preventing diabetes.

### Dosing and Compositions

Active agents of the disclosure are preferably administered orally in a total daily dose of about 0.001 mg/kg/dose to about 100 mg/kg/dose, alternately from about 0.01 mg/kg/dose to about 30 mg/kg/dose. In another embodiment the dose range is from about 0.05 to about 10 mg/kg/day. Alternately about 0.05 to about 1 mg/kg/day is administered. Generally, between about 1 mcg and about 1 gram per day can be administered; alternately between about 1 mcg and about 200 mg can be administered. The use of time-release preparations to control the rate of release of the active ingredient may be preferred. The dose may be administered in as many divided doses as is convenient. Such rates are easily maintained when these compounds are intravenously administered as discussed below.

For the purposes of this disclosure, the compounds may be administered by a variety of means including orally, parenterally, by inhalation spray, topically, or rectally in formulations containing pharmaceutically acceptable carriers, adjuvants and vehicles. The term parenteral as used here includes but is not limited to subcutaneous, intravenous, intramuscular, intraarterial, intradermal, intrathecal and epidural injections with a variety of infusion techniques. Intraarterial and intravenous injection as used herein includes administration through catheters. Administration via intracoronary stents and intracoronary reservoirs is also contemplated. The term oral as used herein includes, but is not limited to sublingual and buccal. Oral administration includes fluid drinks, energy bars, as well as pill formulations.

Oral compositions generally include an inert diluent or an edible carrier. They can be enclosed in gelatin capsules or compressed into tablets. Depending on the specific conditions being treated, pharmaceutical compositions of the present invention for treatment of atherosclerosis or the other elements of metabolic syndrome can be formulated and administered systemically or locally. Techniques for formulation and administration can be found in “Remington: The Science and Practice of Pharmacy” (20th edition, Gennaro (ed.) and Gennaro, Lippincott, Williams & Wilkins, 2000). For oral administration, the agent can be contained in enteric forms to survive the stomach or further coated or mixed to be released in a particular region of the GI tract by known methods. For the purpose of oral therapeutic administration, the active agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL® or corn starch; a lubricant such as magnesium stearate or STEROTES® a glidant such as colloidal silicon dioxide; a sweetening agent such as sucrose or saccharin; or a flavoring agent such as peppermint, methyl salicylate, or orange flavoring.

According to various embodiments, the composition may be in a form selected from the group consisting of tablets, powders, granules, dragées, pellets, pills, and capsules. Tablets may include but are not limited to film-coated tablets, sublingual tablets, and orally disintegrating tablets. Capsules may include but are not limited to hard and soft gelatin capsules.

According to various embodiments, the composition may be formulated, for example, as a tablet or capsule or as a unit dose that may be suspended in a liquid immediately prior to use. The tablet or capsule may have an enteric coating. The enteric coating (and the capsule, if appropriate) may dissolve or disintegrate, preferably rapidly (e.g. up to 5, 10, 15, 20, 30, 60, 120, 240, 300, or 360 minutes or longer), when it reaches alkaline conditions, for example on entering the small intestine.

Alternatively, the tablet or capsule may not have an enteric coating but may disintegrate in the stomach to release an enteric coated composition comprising agents.

Examples of enteric release materials are pH-sensitive polymers which provide an aqueous barrier and do not dissolve or disintegrate in acidic aqueous environs typical of the stomach, but which do dissolve or disintegrate in the higher pH aqueous environs typical of the intestines. The time duration of the disintegration upon reaching a higher pH condition dictates where in the intestine the agent is released.

Dosage unit forms of certain embodiments include enteric coated capsules or tablets, or enteric coated active agent. Other related dosage unit forms active agent encased in hard- or soft-shelled capsules with the shell made of an enteric release material. Another dosage unit form provides active agent embedded in a matrix which is soluble or erodible in the intestines but not in the stomach.

For the pharmaceutical compositions in dosage unit form, each dosage unit form may contain from about 0.1 mg to about 1000 mg of active agent, more typically from about 1 mg to about 500 mg of active agent, more typically still from about 5 mg to about 200 mg of active agent.

In a specific embodiment, a dosage unit form is directed to an enteric coated tablet comprising a tablet core containing active agent surrounded by an enteric coating. Tablet cores area typically made by mixing granular or powdered active agent with a pharmaceutical carrier and compressing the resulting mixture into a tablet core by conventional means. The tablet core is then coated with an enteric release material by conventional means, such as in a pan coater or a fluidized bed coater. Examples of commercially available enteric release materials which may be used to produce dosage unit forms of the present invention include cellulose acetate trimellitate, hydroxypropyl methylcellulose phthalate, carboxymethylethylcellulose, co-polymerized methacrylic acid/methacrylic acid methyl esters such as, for instance, materials known under the trade name EUDRAGIT® L12.5, L100, or EUDRAGIT® S12.5, S100 or similar compounds used to obtain enteric coatings, methacrylic acid copolymers (Eudragit® L, S and L30D from Rohm Pharma GmbH, Darmstadt, West Germany); cellulose acetate phthalate (Aquateric® from FMC Corp., Philadelphia, Pa.); polyvinyl acetate phthalate (Coteric® from Colorcon Inc., West Point, Pa.); and hydroxypropyl methylcellulose phthalate (HP50 and HP55 from Shin-Etsu Chemical Co., Ltd., Tokyo, Japan). The preferred thickness of enteric coating used is sufficient to protect the active agent from exposure in the stomach but disintegrates rapidly in the intestines, preferably in the small intestine, more preferably in the duodenum or jejunum, to expose the active agent, such that it contacts gut cells, preferably serotonin+ enteroendocrine cells in the intestine.

Another dosage unit form embodiment is an enteric coated hard gelatin capsule containing active agent. Active agent is typically mixed with a pharmaceutical carrier and filled into hard gelatin capsule shells. The capsules are then enteric coated using a coating as described for enteric coated tablets above.

Another dosage unit form embodiment is enteric coated granules of active agent. Granules comprising active agent and, preferably, a pharmaceutical carrier are prepared and enterically coated using an enteric coating material as described hereinabove. A dosage unit form of the enteric coated granules is prepared by, preferably blending them with an appropriate pharmaceutical carrier, and compressing them into tablets or filling them into hard gelatin capsule shells by conventional means.

Another dosage unit form embodiment pertains to a soft gelatin capsule containing a solution, suspension or emulsion of active agent. The soft gelatin capsule shell is made of an enteric release material which remains intact in the stomach and prevents exposure of the active agent in the stomach, but which dissolves or disintegrates in the intestines and releases the active agent in the intestine as described above.

Systemic administration can also be by transmucosal means to the intestinal or colon. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) can also be used as pharmaceutically acceptable carriers. These can be prepared according to methods known to those skilled in the art, for example, as described in U.S. Pat. No. 4,522,811.

In an embodiment of the disclosure, the agent can be delivered by long-term, automated drug delivery to the gut using an osmotic pump to infuse a desired dose of the agent for a desired time. Insulin pumps can be adapted to deliver the agent to the gut. The delivery rate of the agent to control glucose intolerance, diabetes types 1 or 2 can be readily adjusted through a large range to accommodate changing insulin requirements of an individual (e.g., basal rates and bolus doses). New pumps permit a periodic dosing manner, i.e., liquid is delivered in periodic discrete doses of a small fixed volume rather than in a continuous flow manner. The overall liquid delivery rate for the device is controlled and adjusted by controlling and adjusting the dosing period. The pump can be coupled with a continuous blood glucose monitoring device and remote unit, such as a system described in U.S. Pat. No. 6,560,471, entitled “Analyte Monitoring Device and Methods of Use.” In such an arrangement, the hand-held remote unit that controls the continuous blood glucose monitoring device could wirelessly communicate with and control both the blood glucose monitoring unit and the fluid delivery device delivering therapeutic agents of the present invention. In certain embodiments, the agent may be administered at a rate of from about 0.3-100 ng/hour, preferably about 1-75 ng/hour, more preferably about 5-50 ng/hour, and even more preferably about 10-30 ng/hour. The agent may be administered at a rate of from about 0.1-100 pg/hr, preferably about 1-75 micrograms/hr, more preferably about 5-50 micrograms/hr, and even more preferably about 10-30 micrograms/hr. It will also be appreciated that the effective dosage of an active agent used for treatment may increase or decrease over the course of a particular treatment. Changes in dosage may result and become apparent from monitoring the level of insulin and/or monitoring glycemia control in a biological sample, preferably blood or serum.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents; such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the disclosure contain the active materials in admixture with excipients suitable for the manufacture of aqueous-suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The pharmaceutical compositions of the disclosure may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain 0.07 to 1.7 mmol (approximately 20 to 500 mg) of active material compounded with an appropriate and convenient amount of carrier material-which may vary from about 5 to about 95% of the total compositions. It is preferred that the pharmaceutical composition be prepared which provides easily measurable amounts for administration.

As noted above, formulations of the disclosure suitable for oral administration may be presented as discrete units such as capsules, cachets or tablets each containing a predetermined amount of the active ingredient, as a powder or granules; as a solution or a suspension in an aqueous or non-aqueous liquid, or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropyl ethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide. slow or controlled release of the active ingredient therein using, for example, hydroxypropyl methylcellulose in varying proportions to provide the desired release profile. Tablets may optionally be provided with an enteric coating, to provide release in parts of the gut other than the stomach. This is particularly advantageous with the compounds of formula 1 when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate. Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

As used herein, pharmaceutically acceptable salts include, but are not limited to: acetate, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydroxymethyl)aminomethane. Pharmaceutically acceptable salts may also include halide salts, such as hydrochloride, hydrobromide, and hydroiodide salts. Additional pharmaceutically acceptable salts are known to those skilled in the art.

### Conjunctive Agents

Exemplary conjunctive agents that may be formulated and/or administered with any form of an active agent as described herein include, but are not limited to, angiotensin- converting enzyme (ACE) inhibitors, aldosterone antagonists, amphetamines, amphetamine like agents, Angiotensin II receptor antagonists, anti-oxidants, aldose reductase inhibitors, biguanides, sorbitol dehydrogenase inhibitors, thiazolidinediones (glitazones), thiazide and thiazide-like diuretics, triglyceride synthesis inhibitors, uric acid lowering agents, e.g., xanthine oxidase inhibitors, fructokinase inhibitors, and combinations thereof.

Exemplary ACE inhibitors include, but are not limited to, Benazepril (Lotensin), Captopril , Enalapril (Vasotec), Fosinopril, Lisinopril (Prinivil, Zestril), Moexipril (Univasc), Perindopril (Aceon), Quinapril (Accupril), Ramipril (Altace), Trandolapril (Mavik), and combinations thereof.

Exemplary aldosterone antagonists include, but are not limited to, Spironolactone, Eplerenone, Canrenone (canrenoate potassium), Prorenone (prorenoate potassium), Mexrenone (mexrenoate potassium), and combinations thereof.

Exemplary amphetamines include, but are not limited to, amphetamine, methamphetamine, methylphenidate, p-methoxyamphetamine, methylenedioxyamphetamine, 2,5-dimethoxy-4- methylamphetamine, 2,4,5-trimethoxyamphetamine, and 3,4- methylenedioxymethamphetamine, N-ethylamphetamine, fenethylline, benzphetamine, and chlorphentermine as well as the amphetamine compounds of Adderall™; actedron; actemin; adipan; akedron; allodene; alpha-methyl-(.+−.)-benzeneethanamine; alpha-methylbenzeneethanamine; alpha-methylphenethylamine; amfetamine; amphate; anorexine; benzebar; benzedrine; benzyl methyl carbinamine; benzolone; beta-amino propylbenzene; beta-phenylisopropylamine; biphetamine; desoxynorephedrine; dietamine; DL-amphetamine; elastonon; fenopromin; finam; isoamyne; isomyn; mecodrin; monophos; mydrial; norephedrane; novydrine; obesin; obesine; obetrol; octedrine; oktedrin; phenamine; phenedrine; phenethylamine, alpha-methyl-; percomon; profamina; profetamine; propisamine; racephen; raphetamine; rhinalator, sympamine; simpatedrin; simpatina; sympatedrine; and weckamine. Exemplary amphetamine-like agents include but are not limited to methylphenidate. Exemplary compounds for the treatment of ADD include, but are not limited to, methylphenidate, dextroamphetamine/amphetamine, dextroamphetamine, and atomoxetine (non-stimulant).

Exemplary Angiotensin II receptor antagonists or angiotensin receptor blockers (ARBs) include, but are not limited to losartan, irbesartan, olmesartan, candesartan, valsartan, and combinations thereof.

Exemplary anti-oxidant compounds include but are not limited to L-ascorbic acid or L- ascorbate (vitamin C), menaquinone (vitamin K 2), plastoquinone, phylloquinone (vitamin K 1), retinol (vitamin A), tocopherols (e.g. , α, β, γ and δ-tocotrienols, ubiquinol, and ubiquione (Coenzyme Q1 0)); and cyclic or polycyclic compounds including acetophenones, anthroquinones, benzoquiones, biflavonoids, catechol melanins, chromones, condensed tannins, coumarins, flavonoids (catechins and epicatechins), hydrolyzable tannins, hydroxycinnamic acids, hydroxybenzyl compounds, isoflavonoids, lignans, naphthoquinones, neolignans, phenolic acids, phenols (including bisphenols and other sterically hindered phenols, aminophenols and thiobisphenols), phenylacetic acids, phenylpropenes, stilbenes and xanthones. Additional cyclic or polycyclic antioxidant compounds include apigenin, auresin, aureusidin, Biochanin A, capsaicin, catechin, coniferyl alcohol, coniferyl aldehyde, cyanidin, daidzein, daphnetin, deiphinidin, emodin, epicatechin, eriodicytol, esculetin, ferulic acid, formononetin, gernistein, gingerol, 3-hydroxybenzoic acid, 4-hydroxybenzoic acid, 3-hydroxycoumarin, juglone, kaemferol, lunularic acid, luteolin, malvidin, mangiferin, 4-methylumbelliferone, mycertin, naringenin, pelargonidin, peonidin, petunidin, phloretin, p-hydroxyacetophenone, (+)-pinoresinol, procyanidin B-2, quercetin, resveratol, resorcinol, rosmaric acid, salicylic acid, scopolein, sinapic acid, sinapoyl-(S)-maleate, sinapyl aldehyde, syrginyl alcohol, telligrandin umbelliferone and vanillin. Antioxidants may also be obtained from plant extracts, e.g., from blackberries, blueberries, black carrots, chokecherries, cranberries, black currants, elderberries, red grapes and their juice, hibiscus, oregano, purple sweet potato, red wine, rosemary, strawberries, tea (e.g., black, green or white tea), and from various plant ingredients as ellagic acid.

Exemplary aldose reductase inhibitors include, but are not limited to, epalrestat, ranirestat, fidarestat, sorbinil, and combinations thereof.

Exemplary biguanides include, but are not limited to, metformin, and less rarely used phenformin and buformin, proguanil, and combinations thereof.

Exemplary thiazolidinediones include, but are not limited to, troglitazone, pioglitazone, ciglitazone, rosiglitazone, englitazone, and combinations thereof. Exemplary sorbitol dehydrogenase inhibitors are disclosed in U.S. Pat. Nos. 6,894,047, 6,570,013, 6,294,538, and US Published Patent Application No. 20050020578, the entirety of which are incorporated by reference herein.

Exemplary thiazide and thiazide-like diuretics include, but are not limited to, benzothiadiazine derivatives, chlortalidone, metolazone, and combinations thereof. Exemplary triglyceride synthesis inhibitors include, but are not limited to, diglyceride acyltransferase 1 (DGAT-1) inhibitors. Exemplary uric acid lowering agents include, but are not limited to, xanthine oxidase inhibitors, such as allopurinol, oxypurinol, tisopurine, febuxostat, inositols (e.g., phytic acid and myo-inositol), fructokinase inhibitors, and combinations thereof.

Exemplary fructokinase inhibitors include, but are not limited to, osthol, alpha mangosteen, luteolin, osthenol, or indazole derivatives (see US Pub No. 2011/0263559) or Pyrimidinopyrimidine derivatives (US2011/0263559). It is appreciated that suitable conjunctive agents for use in the present invention may also comprise any combinations, prodrugs, pharmaceutically acceptable salts, analogs, and derivatives of the above compounds. In one embodiment, the active agent may be administered to the subject along with one or more active agents

It is appreciated by one skilled in the art that when any one or more the active agents described herein are combined with a conjunctive agent or other active agent, the active agent may critically allow for increased efficacy of the agent or allow for reduction of the dose of the other agent that may have a dose-related toxicity associated therewith.

The mode of administration for a conjunctive formulation may be similar to that described above for active agents.

### EXAMPLES

Introduction

The following examples are put forth to provide those of ordinary skill in the art with a complete disclosure and description of how to perform the methods, how to make, and how to use the compositions and compounds disclosed and claimed herein. Efforts have been made to ensure accuracy with respect to numbers (e.g., amounts, temperature, etc.), but some errors and deviations should be accounted for.

The purpose of the following examples is not to limit the scope of the various embodiments, but merely to provide examples illustrating specific embodiments.

### Example 1

A purpose of this example is to demonstrate the synthesis of intermediate compounds useful for producing the compounds described above.

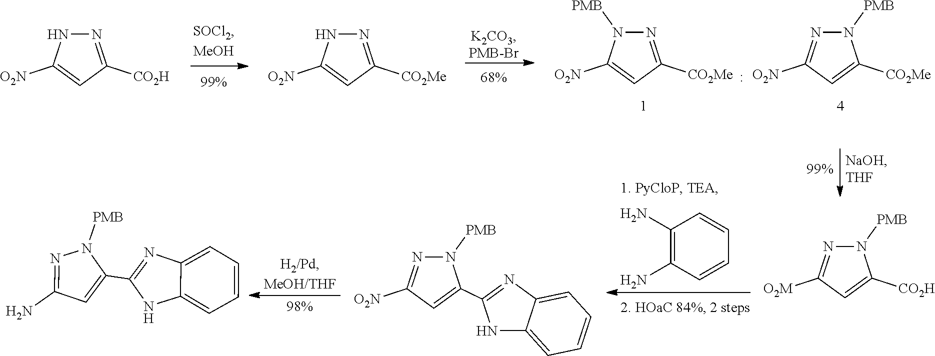

To the solution of 5-nitro-1 H-pyrazole-3-carboxylic acid (4.3 g, 27.5 mmol) in methanol (50 mL) was added thionyl chloride (5.2 mL, 72 mmol) at 0° C. The reaction mixture was refluxed for 3 hr and was concentrated to give methyl ester (4.62 g). This methyl ester (4.62 g, 27 mmol) was dissolved in DMF (30 mL). PMB-Br (6.5 g, 32 mmol) and potassium carbonate (7.45 g, 54 mmol) were added to the solution. The reaction mixture was heated to 75° C. for 3 hr and water (50 mL) was added. The resulting mixture was extracted with ethyl acetate (50 mL×3) and the organic layer was washed with water and brine. The organic layer was dried over sodium sulfate and concentrated to provide crude product (8.46 g). The crude product was recrystalized with ethyl acetate/hexanes (10 mL:25 mL) to provide the pure major regio-isomer (5.37 g, 68%).

Thus obtained compound (5.37 g, 18.45 mmol) was dissolved in methanol/THF (20 mL:40 mL). To the solution was added sodium hydroxide solution (1 M, 27.7 mL, 27.7 mmol) and the reaction mixture was stirred at R.T. overnight and concentrated. HCl solution (2M, 15 mL) was added and the mixture was extracted with ethyl acetate (120 mL). The organic layer was concentrated to provide the acid (5.17 g).

To the solution of the above acid (1.11 g, 4 mmol) and o-phenylenediamine (432 mg, 4 mmol) in dichloromethane (20 mL) was added PyCloP (2.45 g, 5.8 mmol) and TEA (1.12 mL). The resulting mixture was stirred at R.T. overnight. Ethyl acetate (30 mL) and hexanes (30 mL) was added to the reaction mixture and the mixture was washed with aqueous sodium bicarbonate, water (×2) and brine. The organic phase was dried with sodium sulfate and concentrated. Column chromatography with 40% ethyl acetate in hexanes provided the product (1.24 g). To the solution of the product in acetic acid (6 mL) was added potassium acetate (406 mg) and the reaction mixture was stirred at 70° C. for 1 hr until the reaction completed. The solvent was evaporated and the resulting mixture was dissolved in ethyl acetate. The organic layer was washed with aqueous sodium bicarbonate and brine, dried with sodium sulfate and concentrated. Column chromatography with 20% ethyl acetate in hexanes provided the product (84% for 2 steps).

To the above product (1.4 g) in methanol/THF (16 mL:16 mL) was added Pd/C (10%, 150 mg) and the reaction was stirred for 5 hr at 50° C. and then overnight at R.T. under hydrogen atmosphere. The solid was filtered and washed with methanol and the organic solvent was concentrated to provide the aniline (1.16 g).

### Example 2

A purpose of this example is to demonstrate the synthesis of intermediate compounds useful for producing the compounds described above.

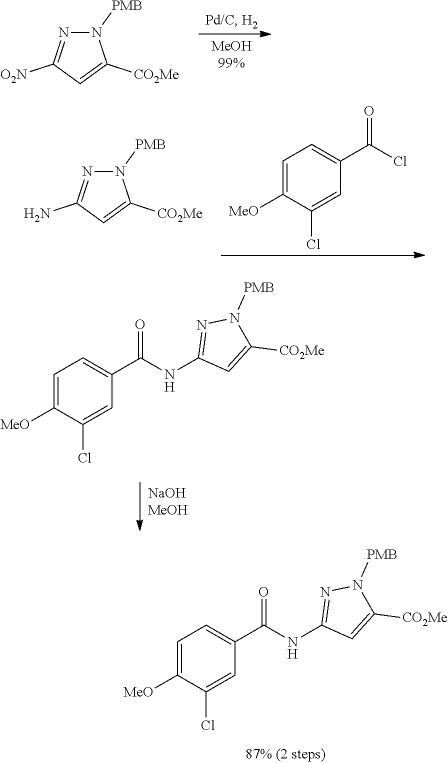

To the nitro compound (1 g, 3.4 mmol) in methanol (10 mL) was added Pd/C (182 mg, 10%, 0.05 equiv.). The resulting mixture was stirred under hydrogen atmosphere overnight. The solid was filtered off and the solvent was removed with rotavapor. The crude product was purified with slica gel using 10% methanol in dichloromethane to recover 460 mg of starting material and provide 480 mg of product (99% based on the recovery of starting material).

3-chloro-4-methoxybenzoid acid (360 mg) was treated with oxalyl chloride (0.42 mL, 2.5 equiv.) and DMF (one drop) in dichloromethane. When there was no bubble evolving, the reaction mixture was concentrated and dissolved in dichloromethane (10 mL) again. To the above amine (480 mg) in DCM (10 mL) and triethylamine (0.77 mL) was added the acyl chloride solution slowly. The resulting mixture was stirred at room temperature overnight and concentrated. The crude product was purified with silica gel column with 5% ethyl acetate in DCM to provide 790 mg product.

To The above product (790 mg) in methanol (10 mL) was added sodium hydroxide solution (2.8 mL, 1 M). The reaction was stirred at room temperature overnight and concentrated. The resulting mixture was acidified with 2N HCl and extracted with ethyl acetate. The organic phase was dried and concentrated to provide the acid (667 mg, yield: 87% for 2 steps).

### Example 3

A purpose of this example is to demonstrate the synthesis of intermediate compounds useful for producing the compounds described above.

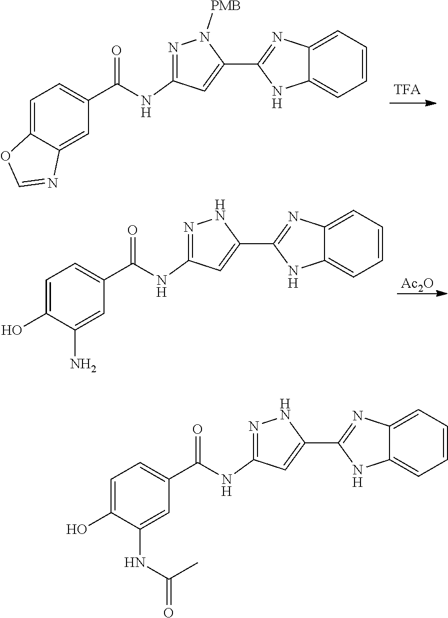

The starting material (10.8 mg) was treated with TFA (2 mL) at 70° C. for 15 minutes. Methanol was added and the solvent was removed under reduced pressure. The resulting mixture was triturated with ethyl acetate and hexanes (2 mL; 1:1) to provide the product (11.3 mg, as TFA salt). LC/MS analysis was used to confirm the product.

To the solution of the above product (5 mg) in DCM (1 mL) was added acetic anhydride (0.02 mL). The reaction was stirred overnight. More Ac₂O was added until the reaction was complete by LC/MS. The solvent was evaporated and the product was triturated with acetate and hexanes (1 mL; 1:1) to provide the product.). LC/MS analysis was used to confirm the product.

### Example 4

A purpose of this example is to demonstrate a general method for synthesis of various compounds described herein from the intermediate compounds described in Examples 1, 2, or 3.

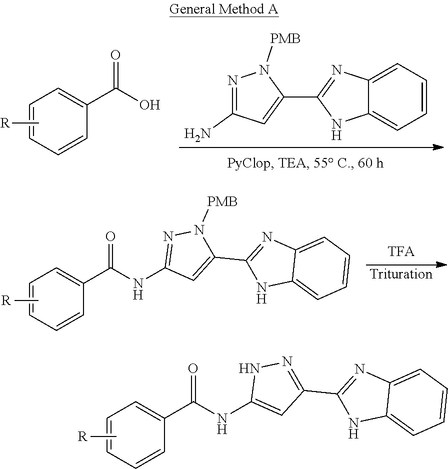

To the mixture of acid (0.047 mmol, 1.0 equiv.) , aniline (15 mg, 0.047 mmol, 1.0 equiv.) and TEA (0.025 mL) in 1,2-dichloroethane (2 mL) was added PyCloP (25.8 mg, 1.3 equiv.). The resulting mixture was stirred at 55° C. for 60 hr. When the reaction was complete, the reaction mixture was loaded to preparative TLC (or silica gel column for large scale) and eluted with 10% methanol in dichloromethane or 20% ethyl acetate in dichloromethane. A typical yield was between 50-90%. Thus obtained product was treated with TFA (1 mL). The TFA solution was heated to 70° C. and kept for 10 min. Methanol was added and the solvent was removed under reduced pressure. The resulting mixture was triturated with ethyl acetate and hexanes (2 mL; 1:1). LC/MS analysis was used to confirm the product.

### Example 5

A purpose of this example is to demonstrate a general method for synthesis of various compounds described herein from the intermediate compounds described in Examples 1, 2, or 3.

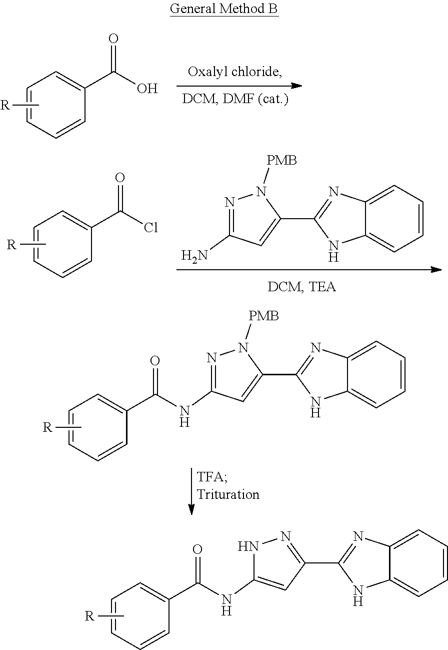

To the mixture of acid (0.047 mmol, 1.0equiv.) in dichloromethane (2 mL) was added oxalyl chloride(0.0082 mL, 0.094 mmol, 2 equiv.) and DMF (0.002 mL). The resulting mixture was stirred at room temperature for 1 h until no bubble evolves. The solvent was removed in vacuo. To the solution of aniline (15 mg, 0.047 mmol, 1.0 equiv.) and TEA (0.025 mL) in dichloromethane (2 mL) was added thus prepared acyl chloride (0.047 mmol, 1.0 equiv.). The resulting mixture was stirred at R.T. for 18 hr. When the reaction was complete, the reaction mixture was loaded to preparative TLC (or silica gel column for large scale) and eluted with 10% methanol in dichloromethane or 20% ethyl acetate in dichloromethane, depending on the product's polarity. A typical yield was between 50-90%. Thus obtained product was treated with TFA (1 mL). The TFA solution was heated to 70° C. and kept for 10 min. Methanol (5 mL) was added and the solvent was removed under reduced pressure. The resulting mixture was triturated with ethyl acetate and hexanes (2 mL; 1:1). LC/MS analysis was used to confirm the product.

### Example 6

A purpose of this example is to demonstrate a general method for synthesis of various compounds described herein from the intermediate compounds described in Examples 1, 2, or 3.

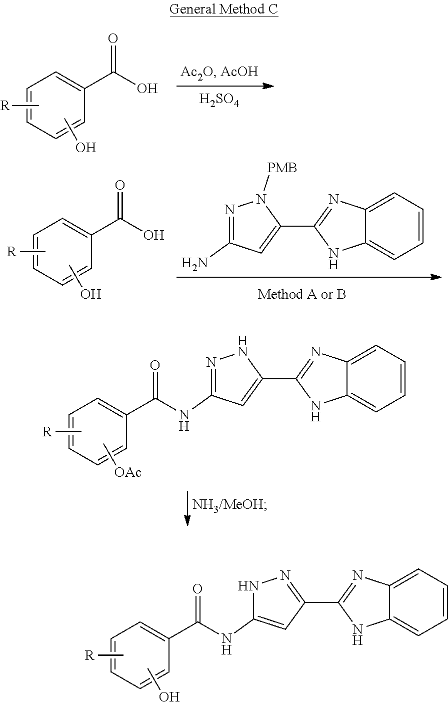

To the mixture of hydroxybenzoic acid (5.8 mmol) and acetic acid (2 mL) were added acetic anhydride (1.64 mL, 17.4 mmol, 3 equiv.) and sulfuric acid (0.01 mL). The resulting mixture was heated to 70° C. and kept for 10 min. Water (20 mL) was added to the cooled reaction mixture. The resulting solid was filtered and washed with water twice and dried to provide the product. Method A or B was used for the coupling and deprotection of PMB. The resulting solid was treated with 7N ammonia in methanol at room temperature for 2 hr. The solvent was removed and the solid was triturated with ethyl acetate and hexanes (2 mL; 1:1) again. A typical yield was between 80-99%. LC/MS analysis was used to confirm the product.

### Example 7

A purpose of this example is to demonstrate a general method for synthesis of various compounds described herein from the intermediate compounds described in Examples 1, 2, or 3.

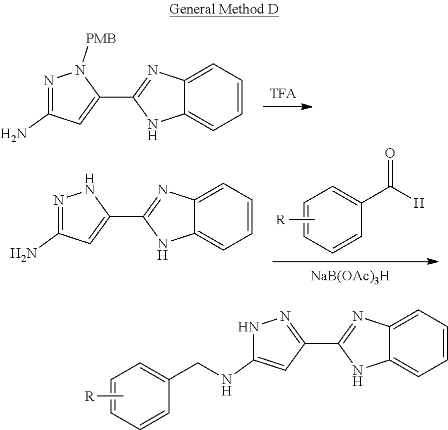

The PMB protected intermediate (0.125 mmol) was treated with TFA (1 mL) at 70° C. for 10 min. Methanol was added to the solution and solvent was evaporated. To the mixture of the resulting amine (TFA salt, 0.063 mmol) in dichloromethane (2 mL) were added the corresponding aldehyde (0.063 mmol, 1 equiv.), sodium triacetoxyborohydride (26 mg, 0.125 mmol, 2 equiv.) and acetic acid (0.004 mL). The resulting mixture was stirred at R.T. overnight. The reaction mixture was dried and loaded to preparative TLC and eluted with 5% methanol in dichloromethane to provide the product. A typical yield was between 60-70% for 2 steps. LC/MS analysis was used to confirm the product.

### Example 8

A purpose of this example is to demonstrate a general method for synthesis of various compounds described herein from the intermediate compounds described in Examples 1, 2, or 3.

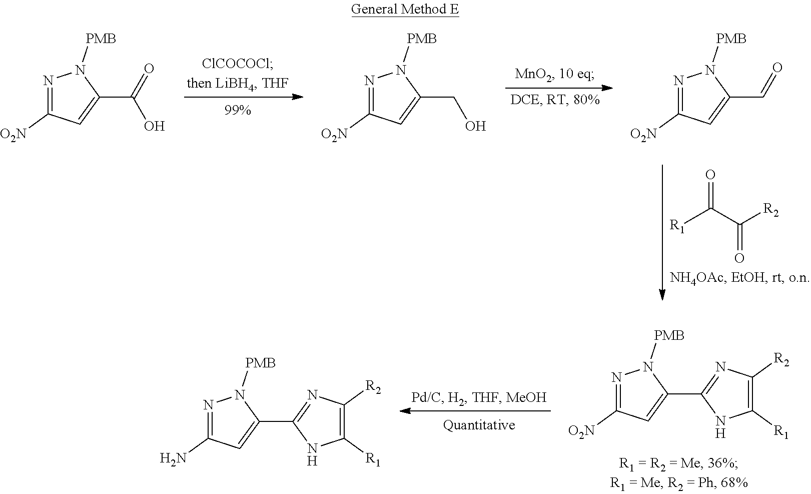

To the solution of acid (500 mg, 1.8 mmol) in DCM (10 mL) was added oxalyl chloride (0.315 mL, 2 equiv.) and DMF (one drop). The reaction was stirred at room temperature until no gas evolves. The solvent was evaporated and the dried acyl chloride was dissolved in THF (20 mL) and was added slowly to the solution of LiBH₄ in THF (2.0M, 3 equiv.). The reaction was stirred at room temperature for 30 minutes and then poured into water. The aqueous layer was extracted with EtOAc (30 mL×2). The organic layer was dried sodium sulfate and concentrated to provide ˜500 mg product.

To the solution of the above alcohol (100 mg) in dichloroethane was added MnO₂ (360 mg, 10 equiv). The resulting mixture was stirred at room temperature overnight. The solid was removed by filtration and the mother liquid was concentrated to provide aldehyde (80 mg).

The above aldehyde (80 mg, 0.31 mmol) was dissolved in ethanol (3 mL). To the ethanol solution was added diketone (15 equiv. for R₁=R₂=Me; or 5 equiv. for R₁=Me, R₂=Ph) and ammonium acetate (12 equiv.). The resulting mixture was stirred overnight and concentrated. The crude product was purified with silica gel (eluent: 10% EtOAc/DCM for for R₁=R₂=Me; and 20% EtOAc/Hexane for R₁=Me, R₂=Ph) to provide the imidazole product (yield: 36% for R₁=R₂=Me; 68% for R₁=Me, R₂=Ph).

Pd/C (15 mg) was added to the above product (0.27 mmol) in mixed solvent (THF 2 mL, MeOH 2 mL) and the resulting mixture was stirred under H2 atmosphere overnight. The solid was removed by filtration and the organic solution was concentrated to provide the product amine in quantitative yield.

### Example 9

A purpose of this example is to demonstrate a general method for synthesis of various compounds described herein from the intermediate compounds described in Examples 1, 2, or 3.

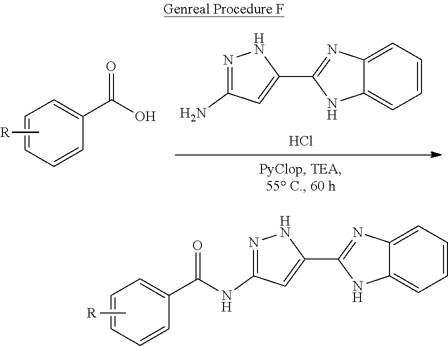

To the mixture of acid (0.047 mmol, 1.0equiv.) , aniline (HCl salt, 15 mg, 0.047 mmol, 1.0 equiv.) and TEA (0.025 mL) in 1,2-dichloroethane (2 mL) was added PyCloP (25.8 mg, 1.3 equiv.). The resulting mixture was stirred at 55° C. for 60 hr. When the reaction was complete, the reaction mixture was loaded to preparative TLC (or silica gel column for large scale) and eluted with 10% methanol in dichloromethane. A typical yield was between 70-75%.

### Example 10

A purpose of this example is to demonstrate a general method for synthesis of various compounds described herein from the intermediate compounds described in Examples 1, 2, or 3.

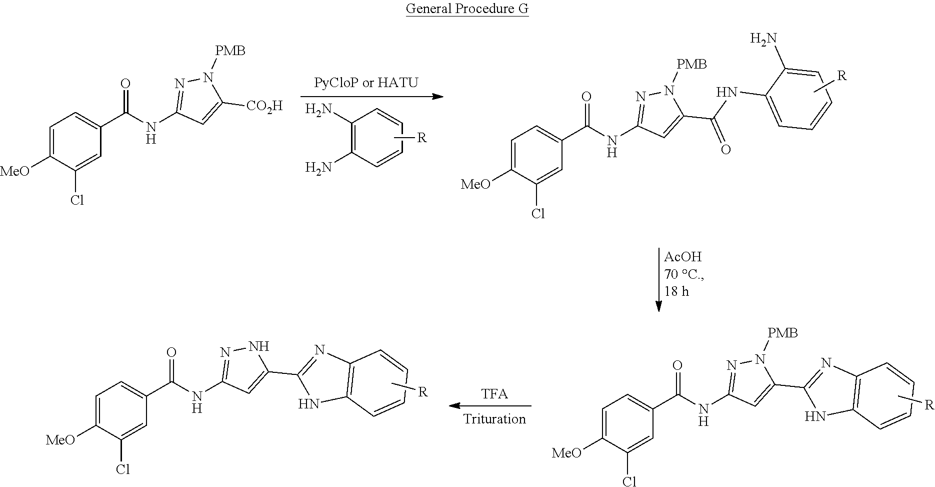

To the mixture of the starting acid (25 mg, 0.06 mmol), diamine (0.06 mmol, 1 equiv.) and triethylamine (0.02 mL) in DCM (1 mL) were added PyCloP (36.7 mg, 0.087 mmol) or HATU (0.087 mmol, General Procedure G-2). The resulting mixture was stirred at room temperature overnight. The reaction mixture was diluted with DCM (20 mL) and was washed with sodium bicarbonate (sat. aq. Solution). The organic phase was dried and concentrated.

To the crude product from above in AcOH (0.5 mL) was added AcOK (7 mg) and the resulting mixture was stirred at 70° C. overnight. The reaction solvent was evaported and the mixture was diluted with EtOAc (20 mL). The organic phase was washed with K₂CO₃ (aq.), NaHCO₃ (aq.) and brine, and then dried and concentrated. The crude product was purified with preparative TLC (eluent: 20% EtOAc in DCM, or 10% MeOH in DCM for polar compounds). The typical yield for the two steps was 45%-85%.

The purified above product was dissolved in TFA. The TFA solution is heated to 70° C. and kept for 10 min. Methanol was added and the solvent was removed under reduced pressure. The resulting solid was triturated with ethyl acetate and hexanes (2 mL; 1:1) to provide the final product. A typical yield for this step was between 80-99%. LC/MS analysis was used to confirm the product.

### Example 11

A purpose of this example is to provide results for compounds synthesized by the general methods (A, B, C, D, E, F, or G) described in Examples 4, 5, 6, 7, 8, 9, or 10 from the intermediate compounds described in Examples 1, 2, or 3.

The activities of FOXO1 inhibitors were determined by a transcriptional reporter assay. HEK293 cells were plated onto 96-well plates with moat at 20000 cells per well in Eagle's Minimum Essential Medium (EMEM) containing 1% fetal bovine serum, and incubated overnight at 37° C. and 5% CO₂. Cells were then transfected using Lipofectamine 3000 (Thermo Fisher Scientific) according to manufacturer's protocol with the following DNA plasmids in each well: (1) 50 ng of pGL4.26 containing 4 tandem copies of an insulin response element (each copy with the sequence of 5′-GCAAAACAAACTTATTTTGAA-3′) (SEQ ID NO: 1) upstream of a firefly luciferase reporter, (2) 5 ng of pcDNA3.1 containing human FOXO1 cDNA with an in-frame FLAG epitope at the 3′ end, (3) 0.5 ng of pRL-CMV encoding constitutively expressed Renilla luciferase. Compounds were then added at varying final concentrations ranging from 50 μM to 1 nM, with final DMSO concentration of 0.5% in each well. Duplicate wells were included for each treatment condition. Cells were incubated for 24 hours at 37° C. and 5% CO₂. Luciferase activities in each well were measured using Dual-Glo Luciferase Assay System (Promega) according to manufacturer's protocol and a plate reader suitable for luminescence detection. Firefly luciferase activity was divided by Renilla luciferase activity to calculate a ratio for each well. The ratio in wells containing cells transfected with all 3 plasmids listed above and receiving only DMSO without compound addition was set to 100%. The ratio in wells containing cells transfected with plasmids (1), (3), and 5 ng of pcDNA3.1 containing the open reading frame of red fluorescent protein (instead of human FOXO1) and treated with DMSO only was set to 0%. The ratio in each well receiving compound treatment was normalized and expressed as a percentage. Data were fit by 4-parameter logistic regression to determine IC₅₀ and maximal inhibition values. Each compound was tested in a minimum of 2 independent experiments. The results are summarized in Table 1.

TABLE 1

| Compound | | Synthetic | MS | |
|---|---|---|---|---|
| # | Structure | method | result | IC₅₀^{a} |
| 1 | 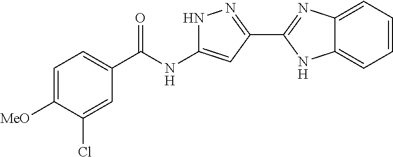 [SMILES: COc1ccc(C(=O)Nc2cc(-c3nc4ccccc4[nH]3)n[nH]2)cc1Cl] | A | 368 [M + H]⁺ | **** |
| 2 | 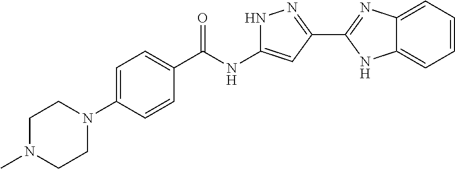 [SMILES: CN1CCN(c2ccc(C(=O)Nc3cc(-c4nc5ccccc5[nH]4)n[nH]3)cc2)CC1] | A | 402 [M + H]⁺ | **** |
| 3 | 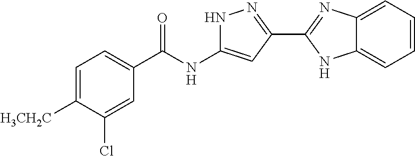 | A | 366 [M + H]⁺ | * |
| 4 | 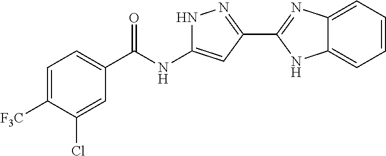 [SMILES: O=C(Nc1cc(-c2nc3ccccc3[nH]2)n[nH]1)c1ccc(C(F)(F)F)c(Cl)c1] | A | 406 [M + H]⁺ | ** |
| 5 | 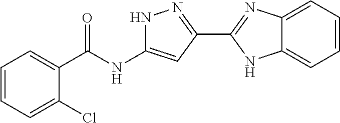 [SMILES: O=C(Nc1cc(-c2nc3ccccc3[nH]2)n[nH]1)c1ccccc1Cl] | A | 338 [M + H]⁺ | * |
| 6 | 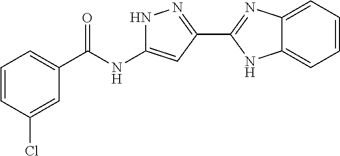 [SMILES: O=C(Nc1cc(-c2nc3ccccc3[nH]2)n[nH]1)c1cccc(Cl)c1] | A | 338 [M + H]⁺ | * |
| 7 | 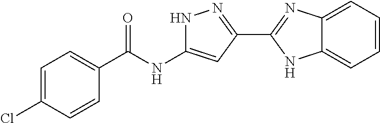 [SMILES: O=C(Nc1cc(-c2nc3ccccc3[nH]2)n[nH]1)c1ccc(Cl)cc1] | A | 338 [M + H]⁺ | * |
| 8 | 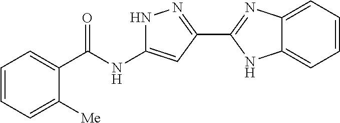 [SMILES: Cc1ccccc1C(=O)Nc1cc(-c2nc3ccccc3[nH]2)n[nH]1] | A | 318 [M + H]⁺ | * |
| 9 | 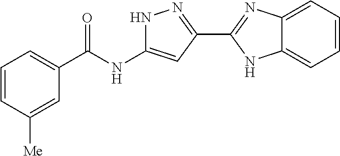 [SMILES: Cc1cccc(C(=O)Nc2cc(-c3nc4ccccc4[nH]3)n[nH]2)c1] | A | 318 [M + H]⁺ | ** |
| 10 | 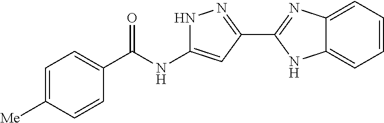 [SMILES: Cc1ccc(C(=O)Nc2cc(-c3nc4ccccc4[nH]3)n[nH]2)cc1] | A | 318 [M + H]⁺ | * |
| 11 | 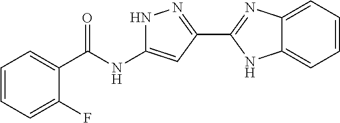 [SMILES: O=C(Nc1cc(-c2nc3ccccc3[nH]2)n[nH]1)c1ccccc1F] | A | 322 [M + H]⁺ | * |
| 12 | 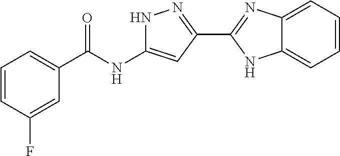 [SMILES: O=C(Nc1cc(-c2nc3ccccc3[nH]2)n[nH]1)c1cccc(F)c1] | A | 322 [M + H]⁺ | * |
| 13 | 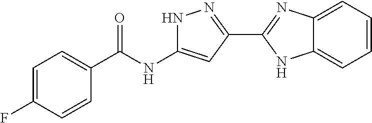 [SMILES: O=C(Nc1cc(-c2nc3ccccc3[nH]2)n[nH]1)c1ccc(F)cc1] | A | 322 [M + H]⁺ | * |
| 14 | 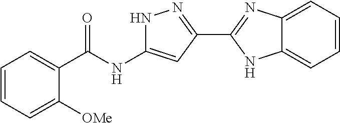 [SMILES: COc1ccccc1C(=O)Nc1cc(-c2nc3ccccc3[nH]2)n[nH]1] | A | 334 [M + H]⁺ | ** |
| 15 | 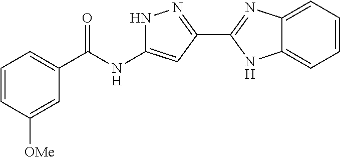 [SMILES: COc1cccc(C(=O)Nc2cc(-c3nc4ccccc4[nH]3)n[nH]2)c1] | A | 334 [M + H]⁺ | *** |
| 16 | 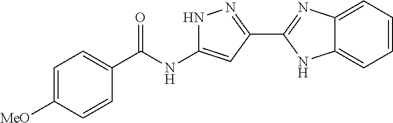 [SMILES: COc1ccc(C(=O)Nc2cc(-c3nc4ccccc4[nH]3)n[nH]2)cc1] | A | 334 [M + H]⁺ | *** |
| 17 | 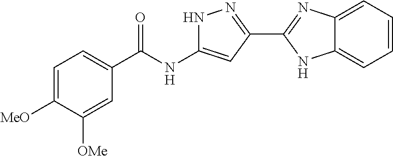 [SMILES: COc1ccc(C(=O)Nc2cc(-c3nc4ccccc4[nH]3)n[nH]2)cc1OC] | A | 364 [M + H]⁺ | **** |
| 18 | 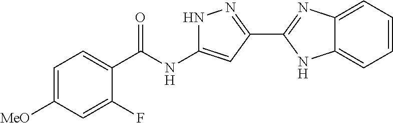 [SMILES: COc1ccc(C(=O)Nc2cc(-c3nc4ccccc4[nH]3)n[nH]2)c(F)c1] | A | 352 [M + H]⁺ | *** |
| 19 | 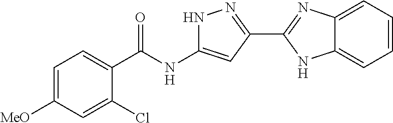 [SMILES: COc1ccc(C(=O)Nc2cc(-c3nc4ccccc4[nH]3)n[nH]2)c(Cl)c1] | A | 368 [M + H]⁺ | *** |
| 20 | 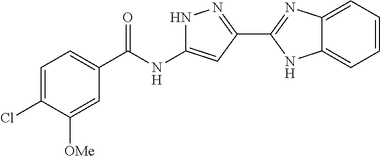 [SMILES: COc1cc(C(=O)Nc2cc(-c3nc4ccccc4[nH]3)n[nH]2)ccc1Cl] | A | 368 [M + H]⁺ | *** |
| 21 | 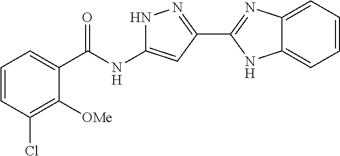 [SMILES: COc1c(Cl)cccc1C(=O)Nc1cc(-c2nc3ccccc3[nH]2)n[nH]1] | A | 368 [M + H]⁺ | ** |
| 22 | 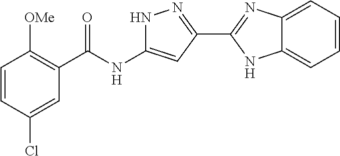 [SMILES: COc1ccc(Cl)cc1C(=O)Nc1cc(-c2nc3ccccc3[nH]2)n[nH]1] | A | 368 [M + H]⁺ | * |
| 23 | 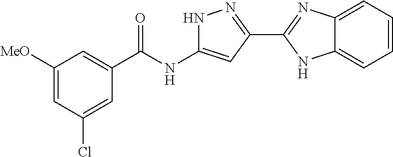 [SMILES: COc1cc(Cl)cc(C(=O)Nc2cc(-c3nc4ccccc4[nH]3)n[nH]2)c1] | A | 368 [M + H]⁺ | ** |
| 24 | 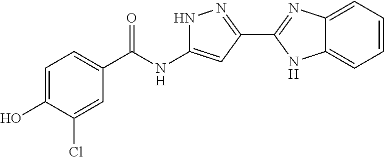 [SMILES: O=C(Nc1cc(-c2nc3ccccc3[nH]2)n[nH]1)c1ccc(O)c(Cl)c1] | C, A | 354 [M + H]⁺ | ** |
| 25 | 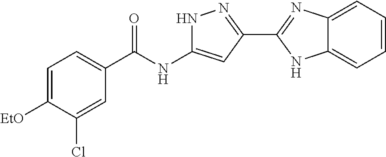 [SMILES: CCOc1ccc(C(=O)Nc2cc(-c3nc4ccccc4[nH]3)n[nH]2)cc1Cl] | A | 382 [M + H]⁺ | **** |
| 26 | 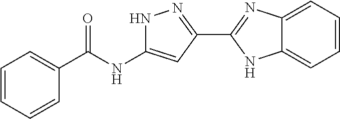 [SMILES: O=C(Nc1cc(-c2nc3ccccc3[nH]2)n[nH]1)c1ccccc1] | B | 304 [M + H]⁺ | * |
| 27 | 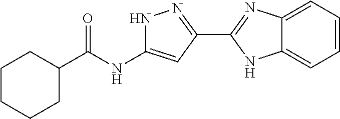 [SMILES: O=C(Nc1cc(-c2nc3ccccc3[nH]2)n[nH]1)C1CCCCC1] | A | 310 [M + H]⁺ | ** |
| 28 | 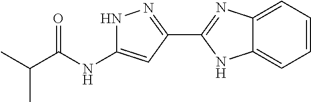 [SMILES: CC(C)C(=O)Nc1cc(-c2nc3ccccc3[nH]2)n[nH]1] | B | 270 [M + H]⁺ | * |
| 29 | 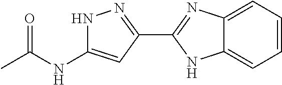 [SMILES: CC(=O)Nc1cc(-c2nc3ccccc3[nH]2)n[nH]1] | B | 242 [M + H]⁺ | * |
| 30 | 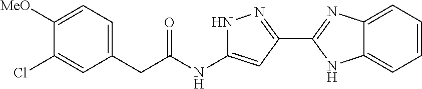 [SMILES: COc1ccc(CC(=O)Nc2cc(-c3nc4ccccc4[nH]3)n[nH]2)cc1Cl] | A | 382 [M + H]⁺ | ** |
| 31 | 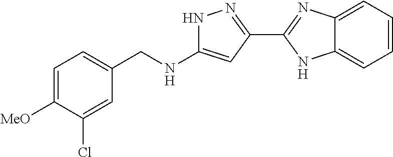 [SMILES: COc1ccc(CNc2cc(-c3nc4ccccc4[nH]3)n[nH]2)cc1Cl] | D | 354 [M + H]⁺ | ** |
| 32 | 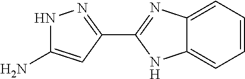 [SMILES: Nc1cc(-c2nc3ccccc3[nH]2)n[nH]1] | D | 200 [M + H]⁺ | * |
| 33 | 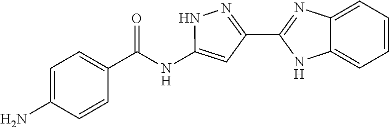 [SMILES: Nc1ccc(C(=O)Nc2cc(-c3nc4ccccc4[nH]3)n[nH]2)cc1] | A | 319 [M + H]⁺ | *** |
| 34 | 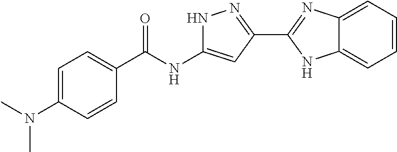 [SMILES: CN(C)c1ccc(C(=O)Nc2cc(-c3nc4ccccc4[nH]3)n[nH]2)cc1] | A | 347 [M + H]⁺ | **** |
| 35 | 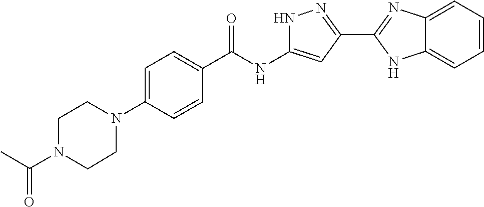 [SMILES: CC(=O)N1CCN(c2ccc(C(=O)Nc3cc(-c4nc5ccccc5[nH]4)n[nH]3)cc2)CC1] | A | 430 [M + H]⁺ | *** |
| 36 | 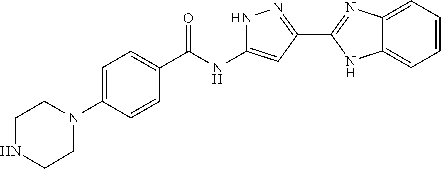 [SMILES: O=C(Nc1cc(-c2nc3ccccc3[nH]2)n[nH]1)c1ccc(N2CCNCC2)cc1] | A | 388 [M + H]⁺ | *** |
| 37 | 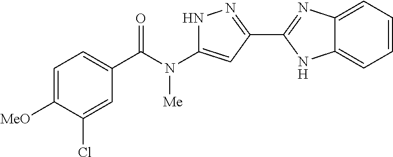 [SMILES: COc1ccc(C(=O)N(C)c2cc(-c3nc4ccccc4[nH]3)n[nH]2)cc1Cl] | B | 382 [M + H]⁺ | * |
| 38 | 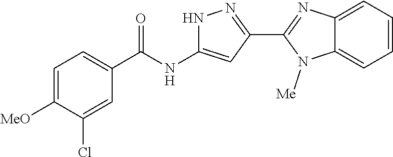 [SMILES: COc1ccc(C(=O)Nc2cc(-c3nc4ccccc4n3C)n[nH]2)cc1Cl] | B | 382 [M + H]⁺ | * |
| 39 | 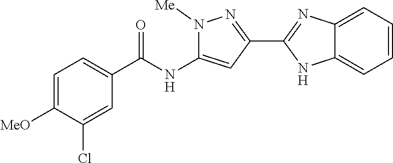 [SMILES: COc1ccc(C(=O)Nc2cc(-c3nc4ccccc4[nH]3)nn2C)cc1Cl] | B | 382 [M + H]⁺ | ** |
| 40 | 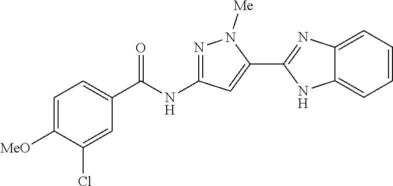 [SMILES: COc1ccc(C(=O)Nc2cc(-c3nc4ccccc4[nH]3)n(C)n2)cc1Cl] | B | 382 [M + H]⁺ | **** |
| 41 | 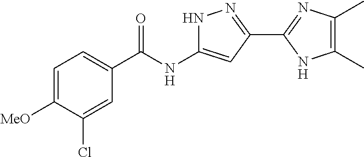 [SMILES: COc1ccc(C(=O)Nc2cc(-c3nc(C)c(C)[nH]3)n[nH]2)cc1Cl] | B, E | 346 [M + H]⁺ | *** |
| 42 | 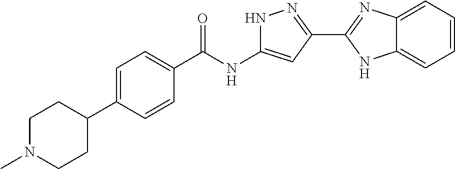 [SMILES: CN1CCC(c2ccc(C(=O)Nc3cc(-c4nc5ccccc5[nH]4)n[nH]3)cc2)CC1] | A | 401 [M + H]⁺ | *** |
| 43 | 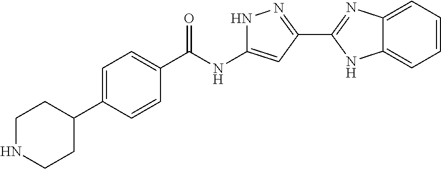 [SMILES: O=C(Nc1cc(-c2nc3ccccc3[nH]2)n[nH]1)c1ccc(C2CCNCC2)cc1] | A | 387 [M + H]⁺ | ** |
| 44 | 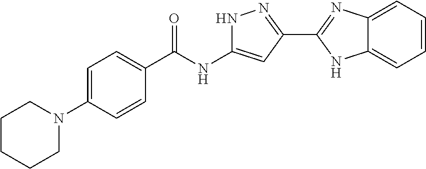 [SMILES: O=C(Nc1cc(-c2nc3ccccc3[nH]2)n[nH]1)c1ccc(N2CCCCC2)cc1] | A | 387 [M + H]⁺ | **** |
| 45 | 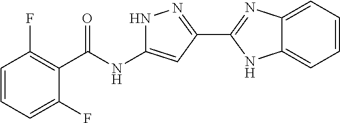 [SMILES: O=C(Nc1cc(-c2nc3ccccc3[nH]2)n[nH]1)c1c(F)cccc1F] | B | 340 [M + H]⁺ | * |
| 46 | 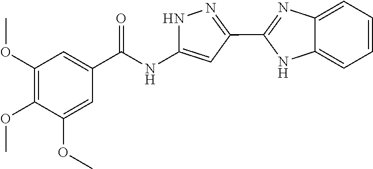 | B | 340 [M + H]⁺ | * |
| 47 | 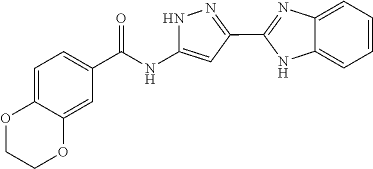 [SMILES: O=C(Nc1cc(-c2nc3ccccc3[nH]2)n[nH]1)c1ccc2c(c1)OCCO2] | B | 362 [M + H]⁺ | *** |
| 48 | 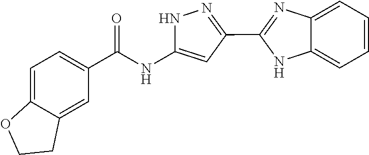 [SMILES: O=C(Nc1cc(-c2nc3ccccc3[nH]2)n[nH]1)c1ccc2c(c1)CCO2] | A | 346 [M + H]⁺ | *** |
| 49 | 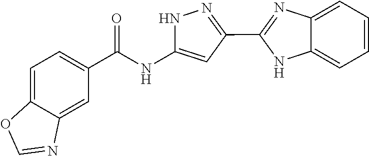 [SMILES: O=C(Nc1cc(-c2nc3ccccc3[nH]2)n[nH]1)c1ccc2ocnc2c1] | F | 345 [M + H]⁺ | ** |
| 50 | 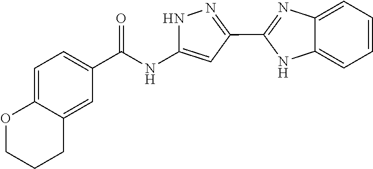 [SMILES: O=C(Nc1cc(-c2nc3ccccc3[nH]2)n[nH]1)c1ccc2c(c1)CCCO2] | B | 360 [M + H]⁺ | ** |
| 51 | 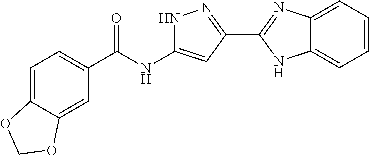 [SMILES: O=C(Nc1cc(-c2nc3ccccc3[nH]2)n[nH]1)c1ccc2c(c1)OCO2] | A | 348 [M + H]⁺ | *** |
| 52 | 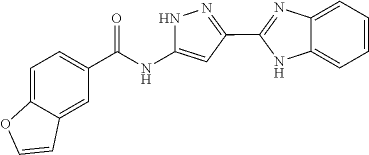 [SMILES: O=C(Nc1cc(-c2nc3ccccc3[nH]2)n[nH]1)c1ccc2occc2c1] | B | 344 [M + H]⁺ | ** |
| 53 | 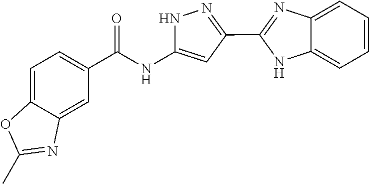 [SMILES: Cc1nc2cc(C(=O)Nc3cc(-c4nc5ccccc5[nH]4)n[nH]3)ccc2o1] | F | 359 [M + H]⁺ | * |
| 54 | 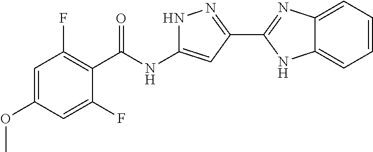 [SMILES: COc1cc(F)c(C(=O)Nc2cc(-c3nc4ccccc4[nH]3)n[nH]2)c(F)c1] | A | 370 [M + H]⁺ | * |
| 55 | 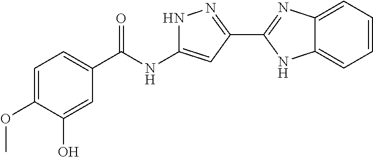 [SMILES: COc1ccc(C(=O)Nc2cc(-c3nc4ccccc4[nH]3)n[nH]2)cc1O] | C, B | 350 [M + H]⁺ | *** |
| 56 | 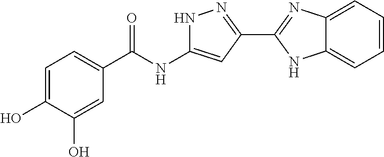 [SMILES: O=C(Nc1cc(-c2nc3ccccc3[nH]2)n[nH]1)c1ccc(O)c(O)c1] | C, B | 336 [M + H]⁺ | * |
| 57 | 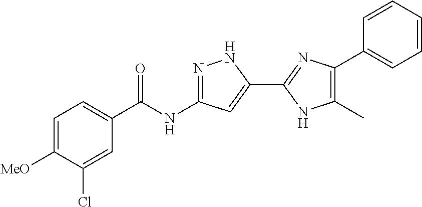 [SMILES: COc1ccc(C(=O)Nc2cc(-c3nc(-c4ccccc4)c(C)[nH]3)[nH]n2)cc1Cl] | B, E | 408 [M + H]⁺ | ** |
| 58 | 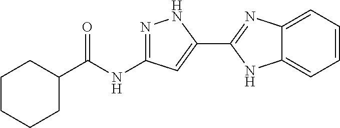 [SMILES: O=C(Nc1cc(-c2nc3ccccc3[nH]2)[nH]n1)C1CCCCC1] | B | 312 [M + H]⁺ | * |
| 59 | 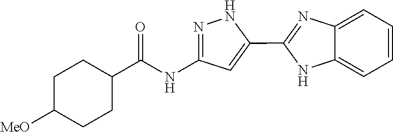 [SMILES: COC1CCC(C(=O)Nc2cc(-c3nc4ccccc4[nH]3)[nH]n2)CC1] | A | 340 [M + H]⁺ | * |
| 60 | 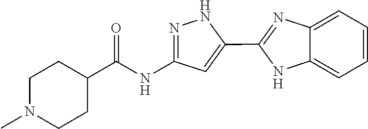 [SMILES: CN1CCC(C(=O)Nc2cc(-c3nc4ccccc4[nH]3)[nH]n2)CC1] | A | 325 [M + H]⁺ | * |
| 61 | 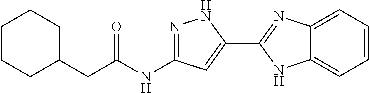 [SMILES: O=C(CC1CCCCC1)Nc1cc(-c2nc3ccccc3[nH]2)[nH]n1] | B | 324 [M + H]⁺ | * |
| 62 | 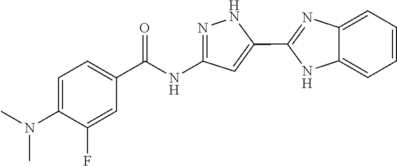 [SMILES: CN(C)c1ccc(C(=O)Nc2cc(-c3nc4ccccc4[nH]3)[nH]n2)cc1F] | A | 365 [M + H]⁺ | *** |
| 63 | 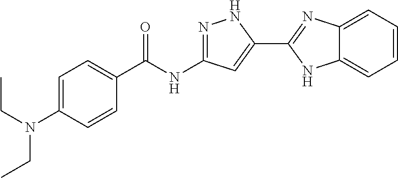 [SMILES: CCN(CC)c1ccc(C(=O)Nc2cc(-c3nc4ccccc4[nH]3)[nH]n2)cc1] | B | 375 [M + H]⁺ | **** |
| 64 | 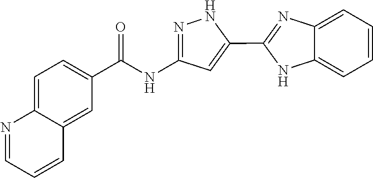 [SMILES: O=C(Nc1cc(-c2nc3ccccc3[nH]2)[nH]n1)c1ccc2ncccc2c1] | A | 355 [M + H]⁺ | ** |
| 65 | 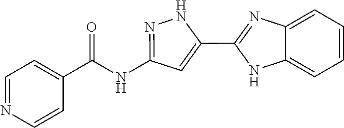 [SMILES: O=C(Nc1cc(-c2nc3ccccc3[nH]2)[nH]n1)c1ccncc1] | A | 305 [M + H]⁺ | * |
| 66 | 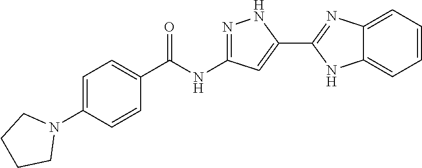 [SMILES: O=C(Nc1cc(-c2nc3ccccc3[nH]2)[nH]n1)c1ccc(N2CCCC2)cc1] | A | 373 [M + H]⁺ | **** |
| 67 | 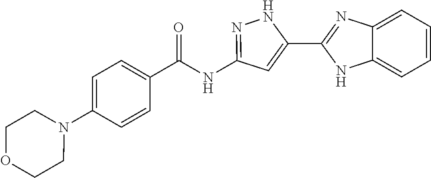 [SMILES: O=C(Nc1cc(-c2nc3ccccc3[nH]2)[nH]n1)c1ccc(N2CCOCC2)cc1] | B | 389 [M + H]⁺ | **** |
| 68 | 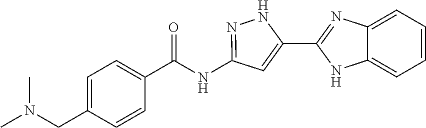 [SMILES: CN(C)Cc1ccc(C(=O)Nc2cc(-c3nc4ccccc4[nH]3)[nH]n2)cc1] | A | 361 [M + H]⁺ | ** |
| 69 | 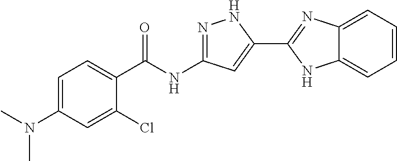 [SMILES: CN(C)c1ccc(C(=O)Nc2cc(-c3nc4ccccc4[nH]3)[nH]n2)c(Cl)c1] | A | 381 [M + H]⁺ | *** |
| 70 | 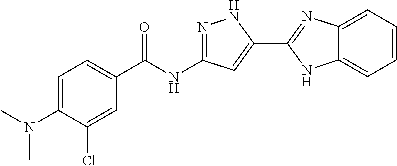 [SMILES: CN(C)c1ccc(C(=O)Nc2cc(-c3nc4ccccc4[nH]3)[nH]n2)cc1Cl] | A | 381 [M + H]⁺ | **** |
| 71 | 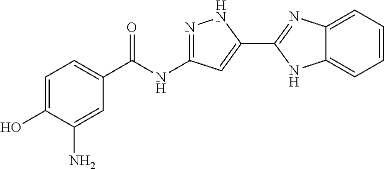 [SMILES: Nc1cc(C(=O)Nc2cc(-c3nc4ccccc4[nH]3)[nH]n2)ccc1O] | I-3 | 335 [M + H]⁺ | * |
| 72 | 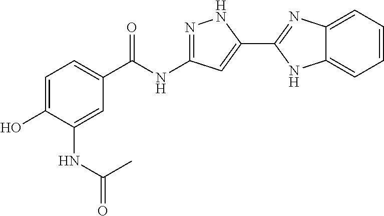 [SMILES: CC(=O)Nc1cc(C(=O)Nc2cc(-c3nc4ccccc4[nH]3)[nH]n2)ccc1O] | I-3 | 377 [M + H]⁺ | * |
| 73 | 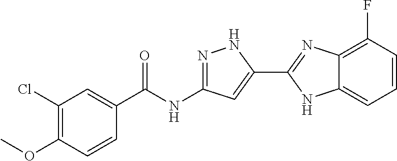 [SMILES: COc1ccc(C(=O)Nc2cc(-c3nc4c(F)cccc4[nH]3)[nH]n2)cc1Cl] | G | 386 [M + H]⁺ | *** |
| 74 | 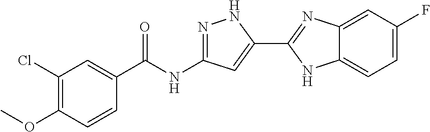 [SMILES: COc1ccc(C(=O)Nc2cc(-c3nc4cc(F)ccc4[nH]3)[nH]n2)cc1Cl] | G | 386 [M + H]⁺ | *** |
| 75 | 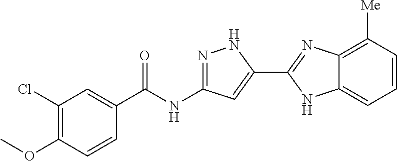 [SMILES: COc1ccc(C(=O)Nc2cc(-c3nc4c(C)cccc4[nH]3)[nH]n2)cc1Cl] | G | 382 [M + H]⁺ | * |
| 76 | 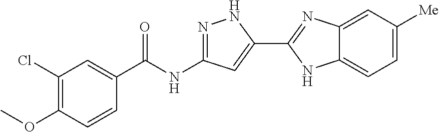 [SMILES: COc1ccc(C(=O)Nc2cc(-c3nc4cc(C)ccc4[nH]3)[nH]n2)cc1Cl] | G | 382 [M + H]⁺ | *** |
| 77 | 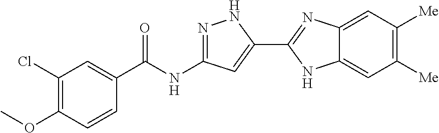 [SMILES: COc1ccc(C(=O)Nc2cc(-c3nc4cc(C)c(C)cc4[nH]3)[nH]n2)cc1Cl] | G | 396 [M + H]⁺ | * |
| 78 | 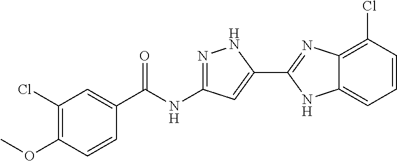 [SMILES: COc1ccc(C(=O)Nc2cc(-c3nc4c(Cl)cccc4[nH]3)[nH]n2)cc1Cl] | G | 402 [M + H]⁺ | *** |
| 79 | 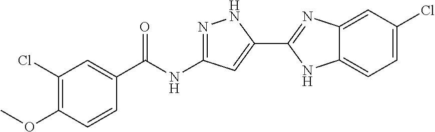 [SMILES: COc1ccc(C(=O)Nc2cc(-c3nc4cc(Cl)ccc4[nH]3)[nH]n2)cc1Cl] | G | 402 [M + H]⁺ | **** |
| 80 | 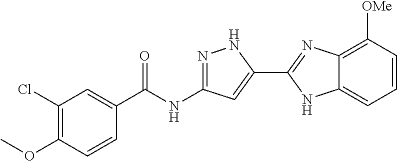 [SMILES: COc1ccc(C(=O)Nc2cc(-c3nc4c(OC)cccc4[nH]3)[nH]n2)cc1Cl] | G | 398 [M + H]⁺ | *** |
| 81 | 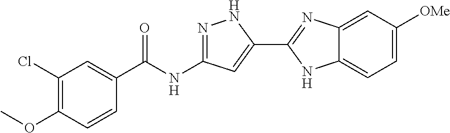 [SMILES: COc1ccc2[nH]c(-c3cc(NC(=O)c4ccc(OC)c(Cl)c4)n[nH]3)nc2c1] | G | 398 [M + H]⁺ | **** |
| 82 | 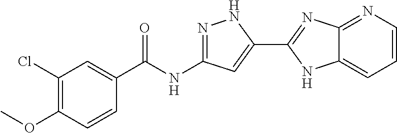 [SMILES: COc1ccc(C(=O)Nc2cc(-c3nc4ncccc4[nH]3)[nH]n2)cc1Cl] | G | 369 [M + H]⁺ | *** |
| 83 | 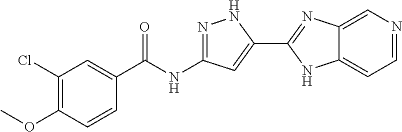 [SMILES: COc1ccc(C(=O)Nc2cc(-c3nc4cnccc4[nH]3)[nH]n2)cc1Cl] | G-2 | 369 [M + H]⁺ | *** |
| 84 | 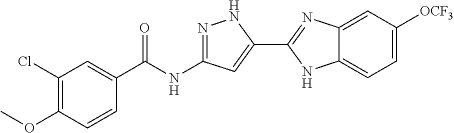 [SMILES: COc1ccc(C(=O)Nc2cc(-c3nc4cc(OC(F)(F)F)ccc4[nH]3)[nH]n2)cc1Cl] | G | 452 [M + H]⁺ | ** |
| 85 | 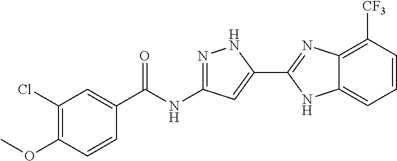 [SMILES: COc1ccc(C(=O)Nc2cc(-c3nc4c(C(F)(F)F)cccc4[nH]3)[nH]n2)cc1Cl] | G | 436 [M +H]⁺ | *** |
| 86 | 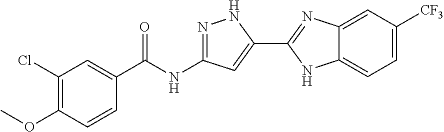 [SMILES: COc1ccc(C(=O)Nc2cc(-c3nc4cc(C(F)(F)F)ccc4[nH]3)[nH]n2)cc1Cl] | G | 436 [M + H]⁺ | *** |

^{a}(active range)
****highly active: IC50 <= 50 nM and maximal inhibition >40%;
***moderately active: IC50 <= 1 μM and maximal inhibition >40%;
**marginally active: 1 μM IC50 <= 10 μM and maximal inhibition >40%;
*inactive: IC50 > 10 μM and/or maximal inhibition <40%;

### Example 12

A purpose of this example was to compare the metabolic stability of compounds 35, 36 and 67 (as identified in Table 1), with a structurally similar compound. Compound 2 (as identified in Table 1, which corresponds with Compound 10 in Langlet et al. Cell. 2017 Nov. 2; 171(4):824-835 was selected as the structurally similar compound. More specifically, the metabolic stability of these compounds was tested in mouse microsomes and also in human hepatic hepatocytes.

A. Metabolic Stability in Mouse Microsomes

An in vitro system comprising CD-1 Mouse liver Microsomes was employed. The incubation conditions and sampling time point of test article and positive control were as follows. Test articles (0.3 μM) and positive controls (verapamil) were incubated in 96-well format. Compound stock solutions were received at 10 mM in DMSO and the Final DMSO % employed in incubation was 0.015%. Incubations were performed 37° C. in 100 mM NaPO₄ Buffer, pH7.4, with 2 mM MgCl₂ at a protein concentration of 0.25 mg/mL, using 1 mM co-factor (NADPH), in a final volume of 500 μL. Incubation time points (+NADPH) at: 0, 5, 15, 30, 45 minutes; 45 min negative control (−NADPH) were employed for recovery assessment.

A solution of the test compound was added to a microsomal preparation in buffer solution at 37° C., followed, after pre-incubation, by either NADPH solution (for the reaction) or buffer (no NADPH, for negative control samples). After the pre-determined incubation time, 50 μL aliquots were removed from the reaction plate and quenched with 200 μL of Stop solution with internal standard (Acetonitrile with 0.1% (v/v) formic acid containing 100 nM labetalol, 20 nM imipramine, and 200 nM diclofenac). The samples were mixed well, centrifuged and submitted for LC-MS/MS analysis. Depletion rate (k_{dep}, min⁻¹) and % of compound remaining are calculated by peak area ratio (PAR) with internal standard at each time point relative to time 0 min. The estimation of Clint(Unscaled) (in pl/min/mg) was performed with the following equation:

$$C {l}_{int , unscaled} = {k}_{dep} \times ( \frac{μ L incubation}{mg microsomal protein} )$$

The results are presented in Table 2:

TABLE 2
Stability in Mouse Microsomes

| | | Mouse microsome | |
|---|---|---|---|
| | | Clᵢₙₜ | % remaining at |
| Compound | Structure | μl/min/mg | 45 min (%) |
| 35 (Table 1) | 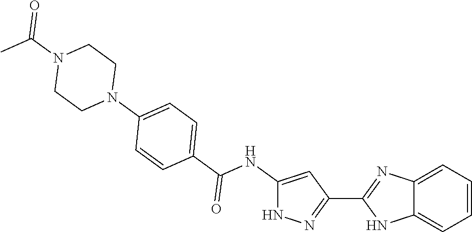 [SMILES: CC(=O)N1CCN(c2ccc(C(=O)Nc3cc(-c4nc5ccccc5[nH]4)n[nH]3)cc2)CC1] | 70 | 44 |
| 36 (Table 1) | 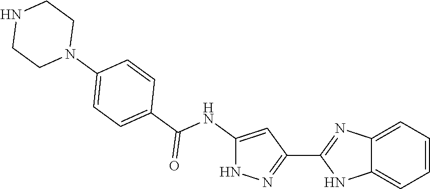 [SMILES: O=C(Nc1cc(-c2nc3ccccc3[nH]2)n[nH]1)c1ccc(N2CCNCC2)cc1] | 41 | 68 |
| 67 (Table 1) | 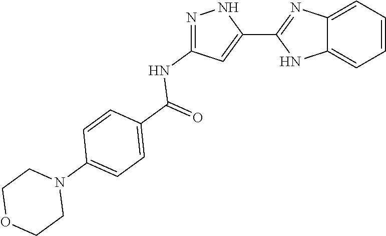 [SMILES: O=C(Nc1cc(-c2nc3ccccc3[nH]2)[nH]n1)c1ccc(N2CCOCC2)cc1] | 52 | 58 |
| 2 (Table 1), (Langlet et al.) (Reference) | 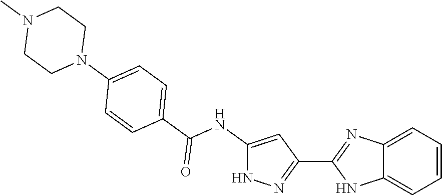 [SMILES: CN1CCN(c2ccc(C(=O)Nc3cc(-c4nc5ccccc5[nH]4)n[nH]3)cc2)CC1] | 206 | 10 |

Clᵢₙₜ: in vitro intrinsic clearance

B. Metabolic Stability in Human Hepatic Hepatocytes

An in vitro system comprising human Hepatocytes was employed. The incubation conditions and sampling time point of test article and positive control were as follows. Test articles (0.3 μM) and positive controls (verapamil) were incubated in a 0.2mL volume in 96-well format. Stock solutions were received at 10 mM in DMSO and the Final DMSO % in incubation was 0.01%. Incubations were performed using cell concentrations of 1×10⁶ cells/mL (200,000 cells/well) in Williams' Medium E with 4 mM L-glutamine Solution: −Cell viability must be >70% as determined by Nexcelom Cellometer. Incubations were initiated by direct addition of test compound and conducted at 37° C./95% humidity/5% CO₂. Incubation plates were shaken on an automated orbital shaker at 600 rpm. Incubation time points were: 0, 15, 30, 60, 90 minutes.

A preparation of diluted hepatocytes (cryopreserved hepatocytes, thawed) was added to a pre-incubated 96-well incubation plate and incubated for 10 minutes in a tissue culture incubator (5% CO₂, 37° C., 95% R.N.). Following this pre-incubation period, the test compound (DMSO stock solution) was added to the incubation plate, which was mixed to initiate the incubation. The reaction plate was sampled (20 μL aliquot) at the pre-determined incubation time points, the sample quenched with 80 μL of Stop solution with internal standard (Acetonitrile with 0.1% (v/v) formic acid containing 100 nM labetalol, 20 nM imipramine, and 200 nM diclofenac) and the samples were mixed well. After quenching the reaction, the reaction mixture was centrifuged and the samples were submitted for LC-MS/MS analysis. Depletion rate (k_{dep}, min⁻¹) and % compound remaining was calculated by peak area ratio (PAR) with internal standard at each time point relative to time 0 min. The estimation of Cl_{int(Unscaled)} (in μl/min/10⁶ cells) was performed with the following equation:

$$C {l}_{int , unscaled} = {k}_{dep} \times ( \frac{μ L incubation}{million hepatocytes} )$$

The results are presented in Table 3:

TABLE 3
Stability in human hepatocytes

| | | Human heptaocyte | |
|---|---|---|---|
| | | Clᵢₙₜ | % remaining |
| Compound | Structure | μl/min/10⁶ cells | at 90 min (%) |
| 36 (Table 1) | 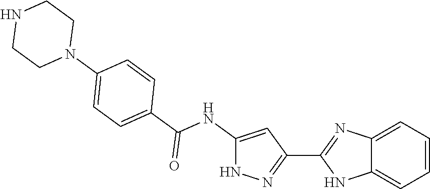 [SMILES: O=C(Nc1cc(-c2nc3ccccc3[nH]2)n[nH]1)c1ccc(N2CCNCC2)cc1] | 2.6 | 110 * |
| 2 (Table 1), (Langlet et al.) (Reference) | 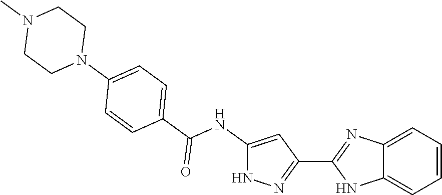 [SMILES: CN1CCN(c2ccc(C(=O)Nc3cc(-c4nc5ccccc5[nH]4)n[nH]3)cc2)CC1] | 18.0 | 22 |

Clᵢₙₜ: in vitro intrinsic clearance
* Criterion for determining signal difference relative to the initial time point is ≥15%

### Example 13

A purpose of this example was to demonstrate the selectivity and effects of Compounds 36 and 67 (as specified in Table 1) as acetylcholinesterase (AChE) inhibitors, compared to the structurally related Compound 2 (as specified in Table 1, which corresponds to Compound 10 in Langlet et al.).

The protein source was Human (recombinant) Expressed in CHO cells. The enzymatic activity was studied. The method employed was the detection of conversion of acetylthiocholine to thiocholine using DTNB. The substrate employed was Acetylthiocholine.

Enzyme and test compound were pre-incubated for 15 minutes at room temp before substrate addition. Acetylthiocholine and DTNB (5,5′-dithiobis-(2-nitrobenzoic acid)) were added and incubated at room temperature for 30 minutes. Signal was detected by measuring absorbance at 405 nm.

Percentage inhibition was calculated using the following formula:

$$\% Inhibition = 100 \% \cdot \frac{\begin{matrix}1 - ( mean Absorbance of test sample - \\ mean Absorbance of vehicle control )\end{matrix}}{\begin{matrix}( mean Absorbance of positive control - \\ mean Absorbance of vehicle control )\end{matrix}}$$

The results are presented in Table 4:

TABLE 4
Selectivity

| | | AChE Inhibitor IC50 |
|---|---|---|
| Compound | Structure | (microM) |
| 36 (Table 1) | 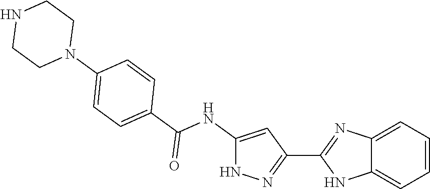 [SMILES: O=C(Nc1cc(-c2nc3ccccc3[nH]2)n[nH]1)c1ccc(N2CCNCC2)cc1] | >10 |
| 67 (Table 1) | 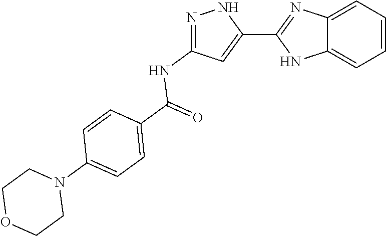 [SMILES: O=C(Nc1cc(-c2nc3ccccc3[nH]2)[nH]n1)c1ccc(N2CCOCC2)cc1] | >10 |
| 2 (Table 1), (Langlet et al.) (Reference) | 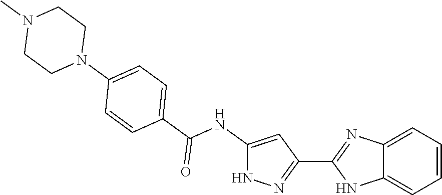 [SMILES: CN1CCN(c2ccc(C(=O)Nc3cc(-c4nc5ccccc5[nH]4)n[nH]3)cc2)CC1] | 1.71 |

As seen in Tables 2 and 3, Compounds 35, 36 and 67 were significantly more stable in mouse microsomes, and/or in human hepatocytes, as compared with the structural analog Compound 10 (Langlet et al.). The replacement of the N-methyl piperazinyl moiety in Compound 10 with an N-acetylpiperazinyl moiety (compound 35), unsubstituted piperazinyl (compound 36), or morpholinyl moiety (compound 67) resulted in high metabolic stability in the tested systems. Moreover, in selectivity studies, compound 10 was found to inhibit AChE (IC₅₀=1.71 μM), as compared with Compounds 36 and 37, which was not observed to inhibit AChE at the concentrations tested (IC 50>10 μM).

### Example 14

A purpose of this example was to compare the metabolic stability of Compounds 20, 24 and 73 (as specified in Table 1), to the structurally related Compound 1 (as specified in Table 1, which corresponds with Compound 9 in Langlet et al.).

The methods employed were the same as in Example 12, but the metabolic stability was tested in Dog and Human Microsomes. The results are presented in Table 5.

As seen in Table 5, Compounds 20, 24 and 73 were significantly more stable in dog and/or human microsomes, as compared with the structural analog Compound 9 (Langlet et al.). The replacement of the 3-chloro-4-methoxyphenyl moiety in Compound 9 with a 4-chloro-3-methoxyphenyl (compound 20), or 3-chloro-4-hydroxyphenyl (compound 24) significantly increased metabolic stability in the tested systems. Moreover, the addition of a fluoro group on the benzimidazole ring of a compound containing a 3-chloro-4-methoxyphenyl moiety (compound 73) significantly increased metabolic stability as compared with the structurally related compound 9, which contains an unsubstituted benzimidazole ring.

TABLE 5
Stability in dog and human microsomes

| | | Dog microsome | | Human microsome | |
|---|---|---|---|---|---|
| | | | % | | % |
| | | | remaining | | remaining |
| | | Clᵢₙₜ | at 45 min | Clᵢₙₜ | at 45 min |
| Compound | Structure | μl/min/mg | (%) | μl/min/mg | (%) |
| 20 (Table 1) | 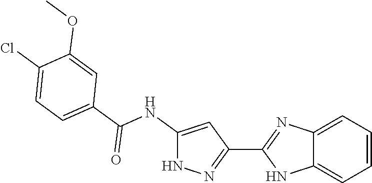 [SMILES: COc1cc(C(=O)Nc2cc(-c3nc4ccccc4[nH]3)n[nH]2)ccc1Cl] | 58 | 53 | 57 | 52 |
| 24 (Table 1) | 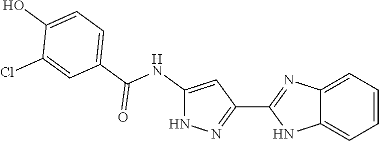 [SMILES: O=C(Nc1cc(-c2nc3ccccc3[nH]2)n[nH]1)c1ccc(O)c(Cl)c1] | <20 | 88 | <20 | 93 |
| 73 (Table 1) | 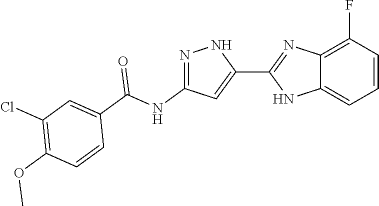 [SMILES: COc1ccc(C(=O)Nc2cc(-c3nc4c(F)cccc4[nH]3)[nH]n2)cc1Cl] | 104 | 31 | 184 | 13 |
| 1 (Table 1), (Langlet et al.) (Reference) | 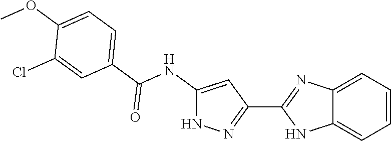 [SMILES: COc1ccc(C(=O)Nc2cc(-c3nc4ccccc4[nH]3)n[nH]2)cc1Cl] | 185 | 12 | 306 | 3.2 |

Clᵢₙₜ: in vitro intrinsic clearance

1. A compound having a structure represented by Formula I: 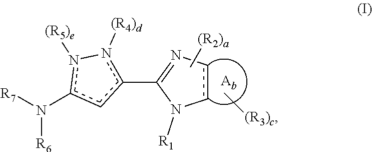
wherein R₁ is selected from the group consisting of H and C₁-C₃ alkyl;
wherein a is selected from the group consisting of 0, 1, and 2;
wherein each R₂ moiety, if present, is independently selected from the group consisting of C₁-C₆ alkyl and C₃-C₁₄ aryl;
wherein b is selected from the group consisting of 0 and 1;
wherein A is a cyclic moiety selected from the group consisting of C₃-C₁₄ aryl and C₃-C₆ heteroaryl;
wherein c is selected from the group consisting of 0, 1, 2, 3, and 4;
wherein each R₃ moiety, if present, is independently selected from the group consisting of H, chlorine (Cl), fluorine (F), C₁-C₃ alkoxy, trifluoromethoxy (OCF₃), trifluoromethyl (CF₃), C₁-C₆ alkyl, and C₃-C₁₄ aryl;
wherein d is selected from the group consisting of 0 and 1;
wherein, if present, R₄ is selected from the group consisting of H, and C₁-C₃ alkyl;
wherein e is selected from the group consisting of 0 and 1;
wherein, if present, R₅ is selected from the group consisting of H, and C₁-C₃ alkyl;
wherein R₆ is selected from the group consisting of H, and C₁-C₃ alkyl;
wherein R₇ is selected from the group consisting of H, a moiety having a structure represented by Formula II, a moiety having a structure represented by Formula III, a moiety having a structure represented by Formula IV, a moiety having a structure represented by Formula V, a moiety having a structure represented by Formula VI, and a moiety having a structure represented by Formula VII, 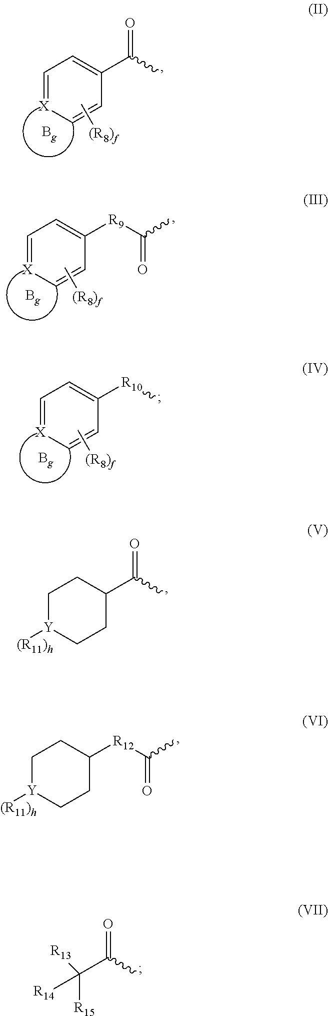
wherein X is selected from the group consisting of C and N;
wherein f is selected from the group consisting of 3, 4, and 5;
wherein each R₈ moiety is independently selected from the group consisting of H, C₁-C₃ alkoxy, chlorine (Cl), fluorine (F), C₁-C₆ alkyl, trifluromethyl (CF₃), hydroxy (OH), an amine moiety, an alkyl amine moiety, an amide moiety, and a heterocyclic amine moiety;
wherein R₉ is a C₁-C₆ alkyl;
wherein R₁₀ is a C₁-C₆ alkyl;
wherein g is selected from the group consisting of 0 and 1;
wherein B is selected from the group consisting of an aryl moiety and a heteroaryl moiety;
wherein h is selected from the group consisting of 0 and 1;
wherein R₁₁ is selected from the group consisting of H, C₁-C₆ alkyl, and C₁-C₃ alkoxy;
wherein R₁₂ is a C₁-C₆ alkyl;
wherein Y is selected from the group consisting of C, N, and O;
wherein R₁₃ is a C₁-C₆ alkyl;
wherein R₁₄ is a C₁-C₆ alkyl; and
wherein R₁₅ is a C₁-C₆ alkyl,
or a pharmaceutically acceptable salt or tautomer thereof,
with the proviso that 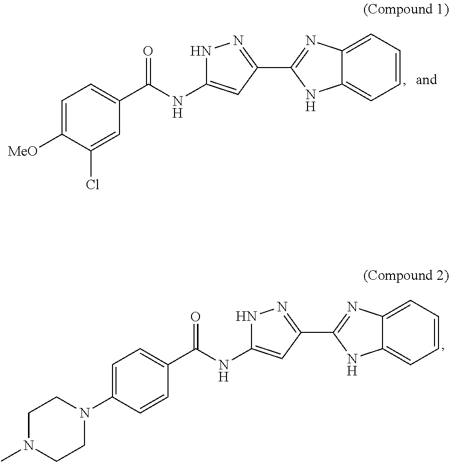 [SMILES: COc1ccc(C(=O)Nc2cc(-c3nc4ccccc4[nH]3)n[nH]2)cc1Cl ; CN1CCN(c2ccc(C(=O)Nc3cc(-c4nc5ccccc5[nH]4)n[nH]3)cc2)CC1]
or tautomers of Compounds (1) and (2) are excluded.

2. The compound according to claim 1, wherein A is a pyridine moiety.

3. The compound according to claim 2, wherein the pyridine moiety having a structure represented by Formula VIII or Formula IX,

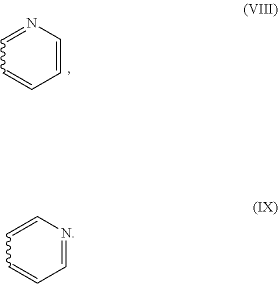 [SMILES: c1ccncc1 ; c1ccncc1]

4. The compound according to claim 1, wherein at least one R₈ is an amine moiety, and wherein the amine moiety has a structure represented by Formula X 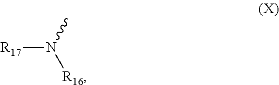
wherein R₁₆ is selected from the group consisting of H and C₁-C₃ alkyl; and
wherein R₁₇ is selected from the group consisting of H and C₁-C₃ alkyl.

5. The compound according to claim 1, wherein at least one R₈ is an alkyl amine moiety, and wherein the alkyl amine moiety has a structure represented by Formula XI 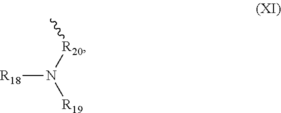
wherein R₁₈ is selected from the group consisting of H and C₁-C₃ alkyl;
wherein R₁₉ is selected from the group consisting of H and C₁-C₃ alkyl; and
wherein R₂₀ is a C₁-C₆ alkyl.

6. The compound according to claim 1, wherein at least one R₈ is an amide moiety, and wherein the amide moiety has a structure represented by Formula XII 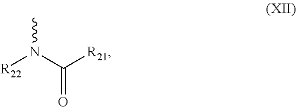
wherein R₂₁ is selected from the group consisting of H and C₁-C₃ alkyl; and
wherein R₂₂ is selected from the group consisting of H and C₁-C₃ alkyl.

7. The compound according to claim 1, wherein at least one R₈ is a heterocyclic amine moiety, and wherein the heterocyclic amine moiety has a structure represented by Formula XIII 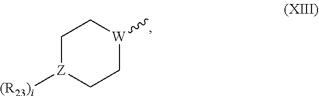
wherein i is selected from the group consisting of 0 and 1;
wherein, if present, R₂₃ is selected from the group consisting of H, C₁-C₆ alkyl, and a ketone moiety;
wherein Z is selected from the group consisting of C, N, and O;
wherein W is selected from the group consisting of C and N.

8. The compound according to claim 1, wherein at least one R₈ is a heterocyclic amine moiety, and wherein the heterocyclic amine moiety has a structure represented by Formula XIV

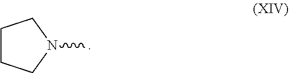

9. The compound according to claim 1, wherein g is 1, wherein B is a heteroaryl moiety, and wherein the heteroaryl moiety is selected from the group consisting of a moiety having a structure represented by Formula XV, 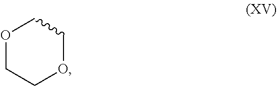
a moiety having a structure represented by Formula XVI, 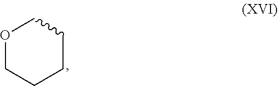
a moiety having a structure represented by Formula XVII, 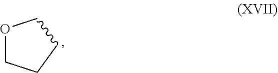
a moiety having a structure represented by Formula XVIII, 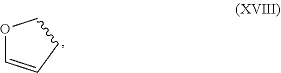
a moiety having a structure represented by Formula XIX, 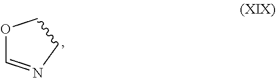
a moiety having a structure represented by Formula XX, 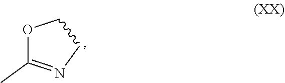
a moiety having a structure represented by Formula XXI, 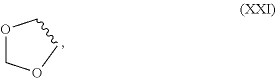
a moiety having a structure represented by Formula XXII,

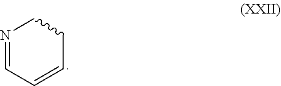

10. The compound according to claim 1,
wherein R₁ is H;
wherein a is 0;
wherein b is 1;
wherein A is a C₆ aryl;
wherein c is 4;
wherein each R₃ moiety is independently selected from the group consisting of H, chlorine, and methoxy;
wherein d is selected from the group consisting of 0 and 1;
wherein, if present, R₄ is selected from the group consisting of H, and C₁-C₃ alkyl;
wherein e is selected from the group consisting of 0 and 1;
wherein, if present, R₅ is H;
wherein R₆ is H;
wherein R₇ is a moiety having a structure represented by Formula II; 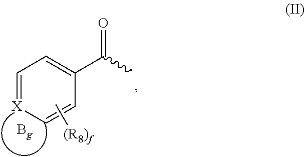
wherein g is 0;
wherein f is 5;
wherein each R₈ moiety is independently selected from the group consisting of H, C₁-C₃ alkoxy, chlorine (Cl), the amine moiety, and a heterocyclic amine moiety.

11. The compound according to claim 10, at least one R₈ moiety is an amine moiety, and
wherein the amine moiety has a structure represented by Formula X 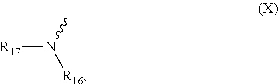
wherein R₁₆ is a C₁-C₂ alkyl; and
wherein R₁₇ is a C₁-C₂ alkyl.

12. The compound according to claim 10, at least one R₈ moiety is a heterocyclic amine moiety, and wherein the heterocyclic amine moiety has a structure represented by Formula XIII 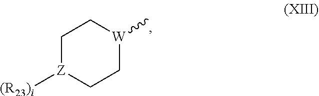
wherein i is selected from the group consisting of 0 and 1;
wherein, if present, R₂₃ is selected from the group consisting of H, and C₁ alkyl;
wherein Z is selected from the group consisting of C, N, and O;
wherein W is N.

13. The compound according to claim 10, at least one R₈ moiety is a heterocyclic amine moiety, and wherein the heterocyclic amine moiety has a structure represented by Formula XIV

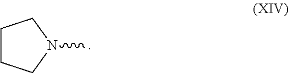

14. The compound according to claim 1, wherein the compound selectively inhibits a Forkhead Box O1 (FOXO1) transcription factor.

15. The compound according to claim 10, wherein the compound has an IC₅₀ less than or equal to 50 nM and a maximal inhibition of FOX01 of greater than 40%.

16. The compound according to claim 1, wherein R₇ is a moiety represented by Formula II, wherein X is C, g is 0 and f is 5, wherein each R₈ moiety is independently selected from the group consisting of H, C₁-C₃ alkoxy, fluorine (F), C₁-C₆ alkyl, trifluromethyl (CF₃), hydroxy (OH), an amine moiety, an alkyl amine moiety, an amide moiety, and a heterocyclic amine moiety.

17. The compound according to claim 16, wherein A is unsubstituted or substituted phenyl and b is 1.

18. The compound according to claim 1, wherein R₇ is a moiety represented by Formula II, wherein X is C, g is 0 and f is 5, wherein each R₈ moiety is independently selected from the group consisting of H, C₂-C₃ alkoxy, chlorine (Cl), fluorine (F), C₁-C₆ alkyl, trifluromethyl (CF₃), hydroxy (OH), an amine moiety, an alkyl amine moiety, and an amide moiety, and a heterocyclic amine moiety.

19. The compound according to claim 1, wherein R₇ is a moiety represented by Formula II, wherein X is C, g is 0 and f is 5, wherein each R₈ moiety is independently selected from the group consisting of H, chlorine (Cl), fluorine (F), C₁-C₆ alkyl, trifluromethyl (CF₃), hydroxy (OH), an amine moiety, an alkyl amine moiety, an amide moiety, and a heterocyclic amine moiety.

20. The compound according to claim 1, wherein R₇ is a moiety represented by Formula II, wherein X is C, g is 0 and f is 5, wherein each R₈ moiety is independently selected from the group consisting of H, C₁-C₃ alkoxy, chlorine (Cl), fluorine (F), C₁-C₆ alkyl, trifluromethyl (CF₃), hydroxy (OH), an amine moiety, and an alkyl amine moiety.

21. The compound according to claim 1, wherein one of R₄ and R₅ is methyl.

22. A method comprising administering to a mammal having a disease or disorder associated with impaired pancreatic endocrine function, a therapeutically effective amount of a compound according to claim 1, or a pharmaceutical composition comprising such compound.

23. The method of claim 22, further comprising co-administering a therapeutically effective amount of a conjunctive agent.

24. The method of claim 23, wherein the conjunctive agent is an inhibitory oligonucleotide targeting Foxo1 expression.

25. The method of claim 22, wherein the disease or disorder is diabetes.

26. The method of claim 22, wherein the compound is orally administered in an enteric form so as to release the therapeutically effective amount in a gut region comprising gut ins- cells or is locally administered directly into or onto the gut region.

27. A method for producing enteroendocrine cells that make and secrete insulin in a mammal, comprising administering to the mammal an effective amount of a compound according to claim 1, or a pharmaceutical composition comprising such compound, wherein administering comprises delivering the compound to gut ins- cells in the mammal in an amount to produce glucose-responsive enteroendocrine cells that make and secrete insulin, and wherein the compound is orally administered in an enteric form so as to release said therapeutically effective amount in a gut region of the mammal that comprises enteroendocrine progenitor cells or is locally administered directly into or onto the gut region.

28. A composition comprising a compound according to claim 1 and a pharmaceutically acceptable carrier.

29. A method for making insulin-producing enteroendocrine cells comprising a) isolating a population of gut ins- cells, b) contacting the population with a compound according to claim 1 to reduce its expression in an amount and under conditions that permit a portion of the population to produce insulin in a glucose-responsive manner, and c) collecting the insulin-producing cells.

30. A pharmaceutical composition comprising a compound according to claim 1.

31. A pharmaceutical composition comprising a compound represented by the structure of formula (I): 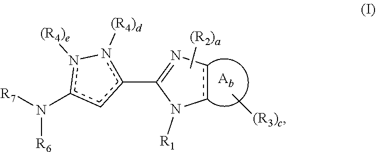
wherein R₁ is selected from the group consisting of H and C₁-C₃ alkyl;
wherein a is selected from the group consisting of 0, 1, and 2;
wherein each R₂ moiety, if present, is independently selected from the group consisting of C₁-C₆ alkyl and C₃-C₁₄ aryl;
wherein b is selected from the group consisting of 0 and 1;
wherein A is a cyclic moiety selected from the group consisting of C₃-C₁₄ aryl and C₃-C₆ heteroaryl;
wherein c is selected from the group consisting of 0, 1, 2, 3, and 4;
wherein each R₃ moiety, if present, is independently selected from the group consisting of H, chlorine (Cl), fluorine (F), C₁-C₃ alkoxy, trifluoromethoxy (OCF₃), trifluoromethyl (CF₃), C₁-C₆ alkyl, and C₃-C₁₄ aryl;
wherein d is selected from the group consisting of 0 and 1;
wherein, if present, R₄ is selected from the group consisting of H, and C₁-C₃ alkyl;
wherein e is selected from the group consisting of 0 and 1;
wherein, if present, R₅ is selected from the group consisting of H, and C₁-C₃ alkyl;
wherein R₆ is selected from the group consisting of H, and C₁-C₃ alkyl;
wherein R₇ is selected from the group consisting of H, a moiety having a structure represented by Formula II, a moiety having a structure represented by Formula III, a moiety having a structure represented by Formula IV, a moiety having a structure represented by Formula V, a moiety having a structure represented by Formula VI, and a moiety having a structure represented by Formula VII, 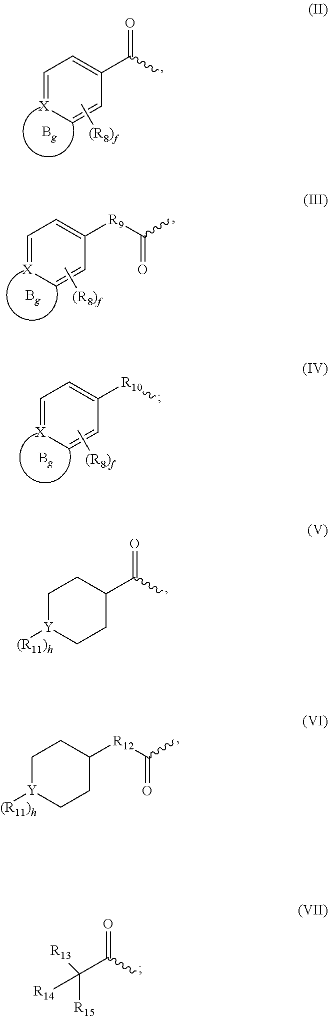
wherein X is selected from the group consisting of C and N;
wherein f is selected from the group consisting of 3, 4, and 5;
wherein each R₈ moiety is independently selected from the group consisting of H, C₁-C₃ alkoxy, chlorine (Cl), fluorine (F), C₁-C₆ alkyl, trifluromethyl (CF₃), hydroxy (OH), an amine moiety, an alkyl amine moiety, an amide moiety, and a heterocyclic amine moiety;
wherein R₉ is a C₁-C₆ alkyl;
wherein R₁₀ is a C₁-C₆ alkyl;
wherein g is selected from the group consisting of 0 and 1;
wherein B is selected from the group consisting of an aryl moiety and a heteroaryl moiety;
wherein h is selected from the group consisting of 0 and 1;
wherein R₁₁ is selected from the group consisting of H, C₁-C₆ alkyl, and C₁-C₃ alkoxy;
wherein R₁₂ is a C₁-C₆ alkyl;
wherein Y is selected from the group consisting of C, N, and O;
wherein R₁₃ is a C₁-C₆ alkyl;
wherein R₁₄ is a C₁-C₆ alkyl; and
wherein R₁₅ is a C₁-C₆ alkyl, or a pharmaceutically acceptable salt or tautomer thereof.

32. The pharmaceutical composition according to claim 30, further comprising at least one pharmaceutically acceptable carrier or excipient.

33. The pharmaceutical composition according to claim 30, wherein the pharmaceutically acceptable carrier or excipient is selected from the group consisting of a diluent, a disintegrating agent, a binder, and a lubricating agent.

34. The pharmaceutical composition according to claim 30, in a form selected from the group consisting of tablets, powders, granules, dragées, pellets, pills, and capsules.
